SEQUENCE LISTING (2 sequences)

SEQ ID NO: 1 - protein, 743 aa, Artificial Sequence
  AAVPHP.B variant capsid
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQTLAVPFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 2 - protein, 743 aa, Artificial Sequence
  AAVPHP.eB variant capsid
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSDGTLAVPFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

(19) United States
(12) Patent Application Publication
(10) Pub. No.: US 2021/0393713 A1
(43) Pub. Date: Dec. 23, 2021

(54) COMPOSITIONS AND METHODS USEFUL FOR TARGETING THE BLOOD-BRAIN BARRIER

(71) Applicant: The Trustees of the University of Pennsylvania, Philadelphia, PA (US)

(72) Inventors: Juliette Hordeaux, Philadelphia, PA (US); James M. Wilson, Philadelphia, PA (US)

(21) Appl. No.: 17/295,255

(22) PCT Filed: Nov. 20, 2019
(86) PCT No.: PCT/US2019/062427

§ 371 Date: May 19, 2021

Related U.S. Application Data
(60) Provisional application No. 62/769,652, filed on Nov. 20, 2018.
(60) Provisional application No. 62/914,035, filed on Oct. 11, 2019.

(51) Int. Cl. A61K 35/761; A61K 47/64; C12N 15/86

(52) U.S. Cl. CPC A61K 35/761; C12N 2750/14132; C12N 15/86; A61K 47/64

(57) ABSTRACT

Compositions and methods for delivering effector entities to the CNS of a subject are provided. Engineered AAV capsids that bind GPI-anchored proteins on the BBB are provided as well as methods for their use, including delivery of gene therapy and effector entities. Also provided, are methods for reducing the infectivity of the CNS by an AAV.

### BACKGROUND OF THE INVENTION

Gene therapy has successfully progressed into the clinic for the treatment of several rare monogenic diseases. A vector platform based on natural isolates of adeno-associated viruses (AAV) has been essential to this success. A natural variant of AAV from human heart muscle called AAV9 (Gao et al. J Virol 78, 6381-6388, 2004; Bell et al. J Clin Invest 121, 2427-2435, 2011) has shown superior distribution following intravenous delivery (Zincarelli et al. Mol Ther 16, 1073-1080, 2008; Duque et al. Mol Ther 17, 1187-1196, 2009; Bevan et al. Mol Ther 19, 1971-1980, 2011). An AAV9-based vector was has also been used to target motor neurons of patients with spinal muscular atrophy resulting in improved motor function and prolonged survival (Mendell et al. N Engl J Med 377, 1713-1722, 2017). Similarly impressive results have been achieved in the treatment of children with a rare inherited myopathy following intravenous delivery of an AAV8 serotype vector isolated from a macaque (Gao et al. Proc Natl Acad Sci USA 99, 11854-11859, 2002) (NCT03199469). Despite improvements in AAV vectors through the isolation of variants such as AAV8 and AAV9, most candidate diseases are outside the reach of successful in vivo gene therapy because of limited delivery to cells of target tissues.

Efficient delivery of gene therapy vectors across the blood-brain barrier (BBB) is crucial to neurological disease therapies. However, adequate delivery levels have yet to be achieved. Various approaches have been pursued to improve the performance of AAV vectors, including engineering capsid variants with enhanced efficiency. One engineering strategy is to create diversity in capsid structure through population mutagenesis and then select preferred candidates by screening the population in cells or animals. The most widely celebrated engineered AAV variant, called AAV-PHP.B, was shown to have superior neurotropic properties (Deverman et al. Nat Biotechnol 34, 204-209, 2016). AAV-PHP.B was identified following the generation of a library of variants by inserting a population of seven amino acid domains into the hypervariable region VIII of AAV9. Variants that target the CNS were identified following intravenous injection into astrocyte-specific CRE recombinase mice on a C57BL/6J background. From this selection emerged AAV-PHP.B, a capsid contains a TLAVPFK domain that shows 50-fold improvement in CNS transduction following intravenous delivery into C57BL/6J mice (Deverman et al. Nat Biotechnol 34, 204-209, 2016). This level of transduction has the potential to expand the utility of AAV gene therapy for human neurological disorders.

There remains a need to improve delivery of gene therapy vectors and therapeutics across the BBB to provide more effective treatment for a variety of CNS conditions.

### SUMMARY OF THE INVENTION

The embodiments described herein relate to compositions and methods to improve delivery of gene therapy vectors and effector entities to the CNS of a subject in need thereof. Methods for altering AAV infectivity of the CNS are also provided.

In one aspect, provided herein is a composition comprising a recombinant AAV having a capsid which comprises a binding partner for a GPI-anchored blood-brain barrier (BBB) ligand and which is conjugated to an effector entity. In certain aspects, the ligand is Ly6E. In another aspect, the ligand is selected from GRA3, ALPL, BST2, EFNA5, NT5E, DPEP2, GPC1, LYPD5, GPC6, CD14, CA4, GPC5, CD59, TFPI, EFNA1, EFNA3, HYAL2, MELTF, ULBP2, EFNA4, CNTN5, BCAN, RECK, CFC1, SEMA7A, PRNP, LY6E, PRND, PLAUR, CD24A, MMP25, ART3, LYPD1, PIBF1, CAPRIN1, GFRA3, GPIHBP1, MACF1, and SEC24B.

In one aspect, the AAV capsid is an empty capsid. In another embodiment, the capsid comprises an AAV vector genome encoding a heterologous gene.

In yet another aspect, the effector entity is a peptide, nucleic acid, siRNA, antibody, antibody fragment, small molecule, lipid nanoparticle, or cytotoxic agent. In one aspect, the AAV capsid and effector entity are conjugated via a linker.

In another embodiment, provided herein is a method for treatment of a neurological disease or disorder in a subject in need thereof comprising contacting the BBB of the subject to an AAV having a capsid which comprises a binding partner for a GPI-anchored BBB ligand and which is conjugated to an effector entity, wherein the capsid binding to the GPI-anchored BBB ligand mediates transport of the effector entity across the BBB. In one embodiment, the ligand is selected from Ly6E, GRA3, ALPL, BST2, EFNA5, NT5E, DPEP2, GPC1, LYPD5, GPC6, CD14, CA4, GPC5, CD59, TFPI, EFNA1, EFNA3, HYAL2, MELTF, ULBP2, EFNA4, CNTN5, BCAN, RECK, CFC1, SEMA7A, PRNP, LY6E, PRND, PLAUR, CD24A, MMP25, ART3, LYPD1, PIBF1, CAPRIN1, GFRA3, GPIHBP1, MACF1, and SEC24B. In another aspect, the neurological disease or disorder is selected from the group consisting of Alzheimer's disease (AD), stroke, dementia, muscular dystrophy (MD), multiple sclerosis (MS), amyotrophic lateral sclerosis (ALS), cystic fibrosis, Angelman's syndrome, Liddle syndrome, Parkinson's disease, Pick's disease, Paget's disease, cancer, a lysosomal storage disorder, and traumatic brain injury. In certain embodiments, the effector entity is a peptide, nucleic acid, siRNA, antibody, antibody fragment, small molecule, or cytotoxic agent. In yet another embodiment, the AAV capsid is conjugated to the effector entity via a linker.

In yet another embodiment, provided herein is a co-therapy for reducing or inhibiting central nervous system uptake of a gene therapy vector having an AAV capsid with a binding partner for a GPI-anchored BBB ligand comprising co-administering with the gene therapy vector an antibody or antibody fragment that binds the BBB ligand. In one aspect, the antibody binds a ligand selected from Ly6E, GRA3, ALPL, BST2, EFNA5, NT5E, DPEP2, GPC1, LYPD5, GPC6, CD14, CA4, GPC5, CD59, TFPI, EFNA1, EFNA3, HYAL2, MELTF, ULBP2, EFNA4, CNTN5, BCAN, RECK, CFC1, SEMA7A, PRNP, LY6E, PRND, PLAUR, CD24A, MMP25, ART3, LYPD1, PIBF1, CAPRIN1, GFRA3, GPIHBP1, MACF1, and SEC24B.

In another aspect, provided herein is a method of engineering an AAV capsid to target the CNS comprising a) identifying an amino acid sequence encoding a peptide fragment that specifically binds a GPI-anchored BBB ligand and b) modifying an AAV HVRVIII site to express the amino acid sequence, wherein the engineered capsid binds a GPI-anchored BBB ligand. In certain embodiments, the ligand is selected from Ly6E, GRA3, ALPL, BST2, EFNA5, NT5E, DPEP2, GPC1, LYPD5, GPC6, CD14, CA4, GPC5, CD59, TFPI, EFNA1, EFNA3, HYAL2, MELTF, ULBP2, EFNA4, CNTN5, BCAN, RECK, CFC1, SEMA7A, PRNP, LY6E, PRND, PLAUR, CD24A, MMP25, ART3, LYPD1, PIBF1, CAPRIN1, GFRA3, GPIHBP1, MACF1, and SEC24B. In another embodiment, the modified AAV is AAV1, AAV3B, or AAV9.

In yet another aspect, provided herein is an engineered AAV capsid obtained by a method of engineering an AAV capsid to target the CNS comprising a) identifying an amino acid sequence encoding a peptide fragment that specifically binds a GPI-anchored BBB ligand and b) modifying an AAV HVRVIII site to express said amino acid sequence, wherein the engineered capsid binds a GPI-anchored BBB ligand.

In one embodiment, provided herein is a method for detectably labeling a CNS target comprising administering an AAV capsid which binds to a GPI-anchored ligand on the BBB and which is conjugated to a detectable effector entity, wherein the AAV capsid upon binding to the GPI-anchored BBB ligand transports the detectable effector entity conjugated thereto across the BBB. In one aspect, the ligand is selected from Ly6E, GRA3, ALPL, BST2, EFNA5, NT5E, DPEP2, GPC1, LYPD5, GPC6, CD14, CA4, GPC5, CD59, TFPI, EFNA1, EFNA3, HYAL2, MELTF, ULBP2, EFNA4, CNTN5, BCAN, RECK, CFC1, SEMA7A, PRNP, LY6E, PRND, PLAUR, CD24A, MMP25, ART3, LYPD1, PIBF1, CAPRIN1, GFRA3, GPIHBP1, MACF1, and SEC24B. In yet another aspect, the detectable effector entity comprises a peptide, nucleic acid, siRNA, antibody, antibody fragment, small molecule, lipid nanoparticle, or cytotoxic agent.

Other aspects and advantages of these compositions and methods are described further in the following detailed description.

### BRIEF DESCRIPTION OF THE DRAWINGS

FIG. 1A-FIG. 1B show the ability of PHP.B to cross the BBB in mice is strain-specific and inherited as a codominant trait. (FIG. 1A) Representative direct GFP fluorescence in the brain of inbred and crossed strains injected IV with 1×10² gc of AAV-PHP.B.CB7.EGFP. The percentage of F2 mice showing intermediate (55.5%), minimal (27.8%) and strong (16.7%) brain transduction is suggestive of Mendelian inheritance. A total of 18 F2 mice were injected. Scale bar 100 μm. (FIG. 1B) Vector genome copies measured by Taqman qPCR in the brain, with average per groups and error bars (standard deviation). Anova Kruska-Wallis test followed by Dunn's multiple comparison test: *p≤0.05, **p≤0.01, ****p≤0.0001 FIG. 2A-FIG. 2D show that the Ly6a gene located on chromosome 15 is associated with the high brain transduction phenotype. (FIG. 2A) WES association study design overview. F1 are generated by crossing the inbred strains C57BL/6J and BALB/cJ, and are all heterozygotes with intermediate brain transduction phenotype. F2 are generated by crossing F1 together, producing a variety of phenotypes and genotypes. WES libraries were generated from genomic DNA isolated from 16 mice (4 F1 and 12 F2) and mapped to the reference genome (GRCm38, C57BL/6J). (FIG. 2B) Manhattan plot, showing genetic variants that are significantly associated (red line, p≤5E-8) with PHP.B transduction across the BBB in mice. The strongest association is observed within the D3 karyotype band (colored in green), which includes the two labeled missense mutations on the Ly6a gene. Genetic confirmation that the top candidate Ly6a (Sca-1) presence is necessary for AAV-PHP.B brain transduction across the BBB in vivo. (FIG. 2C) Representative direct GFP fluorescence in the brain (hippocampus) and liver of inbred WT and Ly6a-null mice after IV administration of 1×10¹² gc of AAV-PHP.B.CB7.EGFP. (FIG. 2D) LY6A immunostaining in the brain cortex of KO and WT mice using a rat monoclonal antibody that recognize both haplotypes (clone D7). LY6A expression is strong in the brain capillaries of C57BL/6J WT mice, weak in the brain capillaries of BALB/cJ WT mice and undetected in the brain of all KO mice. Scale bars (FIG. 2C) 100 μm (FIG. 2D) 50 μm.

FIG. 3A-FIG. 3B show that the Ly6a haplotype determines the AAV-PHP.B ability to cross the BBB in mice. (FIG. 3A) Ly6a haplotypes from a selection of inbred mice (data complied from the mouse genome project, MM10 dbSNP142). (FIG. 3B) AAV-PHP.B brain transduction after systemic administration in inbred mice with the reference Ly6a gene (Ly6^{b} haplotype, C57BL/6J like) and strains with the BALB/cJ like SNPs (Ly6a haplotype). Scale bar 100 μm.

FIG. 4A-FIG. 4C show AAV-PHP.B binds to LY6A proteins. (FIG. 4A) ELISA measurement of the binding of AAV-PHP.B, AAV-PHPeB, AAV9, or an inactive AAV-PHP.B V592G mutant to LY6A proteins. AAV-PHPeB and PHP.B both bind to LY6A, with higher maximal signal for the C57BL/6J variant. AAV9 and AAV-PHP.B V592G do not bind to LY6A. Curves showing average of 3 replicates with standard error of the mean as error bars. (FIG. 4B) AAV.LacZ transduction assay in Ly6a transfected cells. Both Ly6a haplotypes transient expression in HEK293 cells enhance AAV-PHP.B, but not AAV9 transduction while another ly6 family member, ly6c1, has no effect. AAV9 and AAV-PHP.B mediated transduction efficiency within the same transfection conditions is compared using two-way ANOVA followed by a mean-comparison test using Tukey's multiple comparison test (GraphPad Prism). (FIG. 4C) AAV.LacZ transduction assay in presence of anti-LY6A antibody. Enhanced transduction (MOI 10,000) is prevented by preincubation with rat monoclonal anti-LY6A antibody (clone D7, 100 nM, 1 hour at +4° C.). Transduction efficiencies in the presence of different antibodies are compared within the same transfection conditions using two-way ANOVA followed by a mean-comparison test using Tukey's multiple comparison test (GraphPad Prism). Bar graph showing the average of 6 replicates (FIG. 4B) or 8 replicates (FIG. 4C) with standard deviation as error bars.

FIG. 5A-FIG. 5B show AAV-PHP.B cannot cross the BBB in BALB/cJ mice but can transduce brain after intracerebroventricular administration. Direct GFP fluorescence in the brain after (FIG. 5A) intravenous (IV, 1×10¹² gc) or (FIG. 5B) intracerebroventricular (ICV, 1×10¹¹ gc) administration to C57BL/6J and BALB/cJ mice. Scale bars (FIG. 5A) 1 mm; (FIG. 5B) 100 μm.

FIG. 6 shows mutation in the engineered domain of AAV-PHP.B suppresses its ability to cross the BBB. Direct GFP fluorescence in the liver and the brain after IV administration of 1×10² gc of AAV-PHP.B-V592G to C57BL/6J mice. Scale bar 100 μm.

FIG. 7 shows AAV-PHP.eB mediated brain transduction is strain dependent. Direct GFP fluorescence in the brain after intravenous (IV) administration of 1×10¹² gc of AAV-PHP.eB.CB7.EGFP to C57BL6/J and BALBc/J mice. AAV-PHP.eB does not cross the BBB in BALB/cJ. Scale bar 100 μm.

FIG. 8 shows that PHP.B mutants have varying affinity for the Ly6a receptor. Valine 592 in the AAV9-PHP.B capsid, which falls in the middle of the 588-TLAVPFK peptide insertion, was subjected to saturating mutagenesis. Each vector variant was purified and individually tested for binding affinity to Ly6a using SPR. Left to right: Biacore sensograms for high affinity, native affinity, and low affinity variants of AAV9-PHP.B.

FIG. 9 shows the impact of Ly6a binding affinity on cellular transduction. Transduction efficiency of each affinity variant was quantified by expression of beta-galactosidase in HEK293 cells stably expressing Ly6a. Low affinity variants show modest (2-fold) increase in transduction efficiency relative to high affinity variants at low MOI. Any level of binding to the Ly6a receptor is sufficient to boost transduction efficiency >10 fold relative to AAV9.

FIG. 10A-FIG. 10B show the impact of Ly6a binding affinity on biodistribution. (FIG. 10A) Vector with affinity for the Ly6a receptor demonstrates comparable levels of localization to brain tissue. Liver expression of eGFP tracks with biodistribution, where variants with low affinity for Ly6a demonstrate increased localization to and expression in liver tissue. (FIG. 10B) Brain histology indicates that although high and low affinity vectors have similar biodistribution in brain tissue, vectors with low to moderate affinity for Ly6a have improved expression.

FIG. 11 shows valency of PHP.B peptide presentation and effects on cellular transduction. Chimeric capsids were produced by altering the ratios of plasmids encoding either the AAV9 or AAV9-PHP.B capsid gene used during vector production. Transduction efficiency of each chimeric variant was quantified by expression of beta-galactosidase in HEK293 cells stably expressing Ly6a.

FIG. 12 shows multivalent presentation of Ly6a domains is required for binding between soluble Ly6a and PHP.B.

FIG. 13 shows staining of brain sections from non-human primate for the target molecule SEMA7A, which revealed high density expression on brain endothelium.

### DETAILED DESCRIPTION OF THE INVENTION

As described in herein, the present inventors have identified a ligand on the BBB that mediates efficient transport of an AAV vector into the CNS. Although the ligand, LY6A (Sca-1), was previously known, it was believed that the functional role of this GPI-anchored protein was limited to the biology of hematopoiesis. Thus, for the first time the inventors have shown that GPI-anchored proteins can play an important role in facilitating delivery of viral vectors across the BBB. Accordingly, novel compositions and methods for targeting BBB GPI-anchored ligands to deliver gene therapy vectors and/or therapeutic agents to the CNS are disclosed herein.

Unless defined otherwise in this specification, technical and scientific terms used herein have the same meaning as commonly understood by one of ordinary skill in the fields of biology, biotechnology and molecular biology and by reference to published texts, which provide one skilled in the art with a general guide to many of the terms used in the present application. The definitions herein are provided for clarity only and are not intended to limit the claimed invention.

As used herein, the term “subject” means a mammalian animal, including a human, a veterinary or farm animal, a domestic animal or pet, and animals normally used for clinical research. In one embodiment, the subject of these methods and compositions is a human. Still other suitable subjects include, without limitation, murine, rat, canine, feline, porcine, bovine, ovine, non-human primate and others. As used herein, the term “subject” is used interchangeably with “patient”.

The “blood-brain barrier” or “BBB” refers to the physiological barrier between the peripheral circulation and the brain and spinal cord which is formed by tight junctions within the brain capillary endothelial plasma membranes, creating a tight barrier that restricts the transport of molecules into the brain, even very small molecules such as urea (60 Daltons). The BBB within the brain, the blood-spinal cord barrier within the spinal cord, and the blood-retinal barrier within the retina are contiguous capillary barriers within the CNS, and are herein collectively referred to as the blood-brain barrier or BBB. The BBB also encompasses the blood-CSF barrier (choroid plexus) where the barrier is comprised of ependymal cells rather than capillary endothelial cells.

The “central nervous system” or “CNS” refers to the complex of nerve tissues that control bodily function, including the brain and spinal cord.

As used herein “systemic delivery” refers to delivery that leads to a broad biodistribution of a composition described herein within an organism as a result, for example, of the movement of such compositions from one location to another via systemic circulation. Systemic delivery means that a useful, preferably therapeutic, amount of a composition is exposed to most parts of the body. Systemic delivery of the compositions provided herein is preferably obtained by intravenous delivery but may also be achieved by other routes of delivery including oral, inhalation, intranasal, intratracheal, intraarterial, intraocular, intramuscular, and other parental routes.

A “neurological disorder” as used herein refers to a disease or disorder which affects the CNS and/or which has an etiology in the CNS. Exemplary CNS diseases or disorders include, but are not limited to, neuropathy, amyloidosis, cancer, an ocular disease or disorder, viral or microbial infection, inflammation, ischemia, neurodegenerative disease, seizure, behavioral disorders, and a lysosomal storage disorder. Specific examples of neurological disorders include, but are not limited to, neurodegenerative diseases (including, but not limited to, Lewy body disease, post-poliomyelitis syndrome, Shy-Draeger syndrome, olivopontocerebellar atrophy, Parkinson's disease, multiple system atrophy), striatonigral degeneration, tauopathies (including, but not limited to, Alzheimer disease and supranuclear palsy), prion diseases (including, but not limited to, bovine spongiform encephalopathy, scrapie, Creutzfeldt-Jakob syndrome, kuru, Gerstmann-Straussler-Scheinker disease, chronic wasting disease, and fatal familial insomnia), bulbar palsy, motor neuron disease, and nervous system hetero degenerative disorders (including, but not limited to, Canavan disease, Huntington's disease, neuronal ceroid-lipofuscinosis, Alexander's disease, Tourette's syndrome, Menkes kinky hair syndrome, Cockayne syndrome, Halervorden-Spatz syndrome, lafora disease, Rett syndrome, hepatolenticular degeneration, Lesch-Nyhan syndrome, and Unverricht-Lundborg syndrome), dementia (including, but not limited to, Pick's disease, and spinocerebellar ataxia), and cancers affecting the CNS, which include, but are not limited to, glioma, glioblastoma multiforme, meningioma, astrocytoma, acoustic neuroma, chondroma, oligodendroglioma, medulloblastomas, ganglioglioma, Schwannoma, neurofibroma, neuroblastoma, and extradural, intramedullary or intradural tumors.

Viral or microbial infections of the CNS include, but are not limited to, infections by viruses (i.e., influenza, HIV, poliovirus, rubella), bacteria (i.e., Neisseria sp., Streptococcus sp., Pseudomonas sp., Proteus sp., E. coli, S. aureus, Pneumococcus sp., Meningococcus sp., Haemophilus sp., and Mycobacterium tuberculosis) and other microorganisms such as fungi (i.e., yeast, Cryptococcus neoformans), parasites (i.e., Toxoplasma gondii) or amoebas resulting in CNS pathophysiologies including, but not limited to, meningitis, encephalitis, myelitis, vasculitis and abscess, which can be acute or chronic.

An “imaging agent” as used herein is a compound that has one or more properties that permit its presence and/or location to be detected directly or indirectly. Examples of such imaging agents include proteins and small molecule compounds incorporating a labeled entity that permits detection. A “detectable label” is a marker used for detection or imaging. Examples of such labels include: a radiolabel, a fluorophore, a chromophore, or an affinity tag. In one embodiment, the label is a radiolabel used for medical imaging, for example tc99m or iodine-123, or a spin label for nuclear magnetic resonance (NMR) imaging (also known as magnetic resonance imaging, MRI), such as iodine-123, iodine-131, indium-111, fluorine-19, carbon-13, nitrogen-15, oxygen-17, gadolinium, manganese, iron, etc.

The term “cytotoxic agent” as used herein refers to a substance that inhibits or prevents a cellular function and/or causes cell death or destruction. Cytotoxic agents include, but are not limited to, radioactive isotopes (e.g., At, 1-131, 1-125, Y-90, Re-186, Re-188, Sm-153, Bi-212, P-32, Pb-212 and radioactive isotopes of Lu); chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof.

As used herein, the term “treatment,” and variations thereof such as “treat” or “treating,” refers to clinical intervention in an attempt to alter the natural course of the individual being treated, and can be performed either for prophylaxis or during the course of clinical pathology. Desirable effects of treatment include, but are not limited to, preventing or reducing the occurrence or recurrence of disease, alleviation of symptoms, diminishment of any direct or indirect pathological consequences of the disease, decreasing the rate of disease progression, amelioration or palliation of the disease state, and remission or improved prognosis. In some embodiments, effectors described herein are used to delay development of a disease or to slow the progression of a disease. Likewise, “treating cancer” can be described by a number of different parameters including, but not limited to, reduction in the size of a tumor in an animal having cancer, reduction in the growth or proliferation of a tumor in an animal having cancer, preventing, inhibiting, or reducing the extent of metastasis, and/or extending the survival of an animal having cancer compared to control.

As used herein for the described methods and compositions, the term “antibody” refers to an intact immunoglobulin having two light and two heavy chains or fragments thereof capable of binding to a biomarker protein or a fragment of a biomarker protein. Thus, a single isolated antibody or an antigen-binding fragment thereof may be a monoclonal antibody, a synthetic antibody, a recombinant antibody, a chimeric antibody, a humanized antibody, a human antibody, or a bi-specific antibody or multi-specific construct that can bind two or more antigens. As used herein, the term “antibody” may also refer to an antibody fragment.

The term “monoclonal antibody” as used herein refers to an antibody obtained from a population of substantially homogeneous antibodies, i.e., the individual antibodies comprising the population are identical and/or bind the same epitope, except for possible variants that may arise during production of the monoclonal antibody, such variants generally being present in minor amounts. In contrast to polyclonal antibody preparations that typically include different antibodies directed against different determinants (epitopes), each monoclonal antibody is directed against a single determinant on the antigen. In addition to their specificity, the monoclonal antibodies are advantageous in that they are uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al, Nature, 256:495 (1975), or may be made by recombinant DNA methods (see, e.g., U.S. Pat. No. 4,816,567). The monoclonal antibodies may also be isolated from phage antibody libraries using the techniques described in Clackson et al, Nature, 352:624-628 (1991) and Marks et al, J. Mol. Biol, 222:581-597 (1991), for example. Specific examples of monoclonal antibodies herein include chimeric antibodies, humanized antibodies, and human antibodies, including antigen-binding fragments thereof. The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al, Proc. Natl. Acad. Sci. USA, 8 1:6851-6855 (1984)). Chimeric antibodies of interest herein include “primatized” antibodies comprising variable domain antigen-binding sequences derived from a non-human primate {e.g. Old World Monkey, such as baboon, rhesus or cynomolgus monkey) and human constant region sequences (U.S. Pat. No. 5,693,780).

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a nonhuman species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FRs are those of a human immunoglobulin sequence, except for FR substitution(s) as noted above. The humanized antibody optionally also will comprise at least a portion of an immunoglobulin constant region, typically that of a human immunoglobulin. For further details, see Jones et al, Nature 321:522-525 (1986); Riechmann et al, Nature 332:323-329 (1988); and Presta, Curr. Op. Struct. Biol 2:593-596 (1992).

The term “antibody fragment” as used herein for the described methods and compositions refers to less than an intact antibody structure having antigen-binding ability. Examples of antibody fragments include Fab, Fab′, F(ab′)₂, and Fv fragments; diabodies; linear antibodies; single-chain antibody molecules such as e.g. single chain Fab, scFv, and multispecific antibodies formed from antibody fragments. The “Single chain Fab” format is e.g. described in Hust M. et al. BMC Biotechnol. 2007 Mar. 8; 7:14. scFV constructs include complementary scFvs produced as a single chain (tandem scFvs) or bispecific tandem scFvs.

As used herein, “specifically binding,” “binds specifically to,” “specific binding” refer, for example, to an antibody selectively or preferentially binding to an antigen. For example, with respect to an antibody or capsid described herein and the methods of use thereof, specifically binding” refers to preferential binding refers to the ability of the antibody or capsid to bind one or more epitopes of an antigen or binding partner of interest without substantially recognizing and binding other molecules in a sample or environment containing a mixed population of antigens. Specific binding interactions are mediated by one or, typically, more noncovalent bonds between the binding molecules or binding partners. In certain aspects described herein, a binding partner is a capsid protein (or portion thereof) or a molecule conjugated to a capsid protein (such as an antibody) that specifically binds to a target ligand (i.e., a GPI-anchored protein on the BBB). In one embodiment, the GPI-anchored protein that is targeted is a member of the Ly6A family. In certain embodiments, the GPI-anchored protein is selected from Ly6E, GRA3, ALPL, BST2, EFNA5, NT5E, DPEP2, GPC1, LYPD5, GPC6, CD14, CA4, GPC5, CD59, TFPI, EFNA1, EFNA3, HYAL2, MELTF, ULBP2, EFNA4, CNTN5, BCAN, RECK, CFC1, SEMA7A, PRNP, LY6E, PRND, PLAUR, CD24A, MMP25, ART3, LYPD1, PIBF1, CAPRIN1, GFRA3, GPIHBP1, MACF1, and SEC24B.

Various embodiments in the specification are presented using “comprising” language, which is inclusive of other components or method steps. When “comprising” is used, it is to be understood that related embodiments include descriptions using the “consisting of” terminology, which excludes other components or method steps, and “consisting essentially of” terminology, which excludes any components or method steps that substantially change the nature of the embodiment or invention.

The terms “a” or “an” refers to one or more, for example, “a ligand” is understood to represent one or more ligands. As such, the terms “a” (or “an”), “one or more,” and “at least one” are used interchangeably herein.

The term “about” as used herein when referring to a measurable value such as an amount, a temporal duration, and the like, is meant to encompass variations of up to +10% from the specified value; as such variations are appropriate to perform the disclosed methods. Unless otherwise indicated, all numbers expressing quantities of ingredients, properties such as molecular weight, reaction conditions, and so forth used in the specification and claims are to be understood as being modified in all instances by the term “about.”

The terms “compound”, “composition”, or “substance” as used herein may be used interchangeably to discuss the therapeutic composition.

With regard to the description of the inventions provided herein, it is intended that each of the compositions described, is useful, in another embodiment, in the methods of the invention. In addition, it is also intended that each of the compositions herein described as useful in the methods, is, in another embodiment, itself an embodiment of the invention.

In one aspect, an a recombinant AAV having a capsid conjugated to an effector entity is provided. The AAV capsids described herein have a binding partner that specifically binds to a GPI-anchored ligand on the BBB to promote delivery of the conjugate, including effector entity, into the CNS. In one embodiment, the GPI-anchored ligand is Ly6E. Additional conjugates provided herein include AAV capsids that specifically bind other GPI-anchored proteins on the BBB, including, but not limited to, GRA3, ALPL, BST2, EFNA5, NT5E, DPEP2, GPC1, LYPD5, GPC6, CD14, CA4, GPC5, CD59, TFPI, EFNA1, EFNA3, HYAL2, MELTF, ULBP2, EFNA4, CNTN5, BCAN, RECK, CFC1, SEMA7A, PRNP, LY6E, PRND, PLAUR, CD24A, MMP25, ART3, LYPD1, PIBF1, CAPRIN1, GFRA3, GPIHBP1, MACF1, and SEC24B.

The term “AAV” as used herein refers to the dozens of naturally occurring and available adeno-associated viruses, as well as artificial AAVs. An adeno-associated virus (AAV) viral vector is an AAV DNase-resistant particle having an AAV protein capsid into which is packaged nucleic acid sequences for delivery to target cells. An AAV capsid is composed of 60 capsid (cap) protein subunits, VP1, VP2, and VP3, that are arranged in an icosahedral symmetry in a ratio of approximately 1:1:10 to 1:1:20, depending upon the selected AAV. Various AAVs may be selected as sources for capsids of AAV viral vectors as identified above. See, e.g., US Published Patent Application No. 2007-0036760-A1; US Published Patent Application No. 2009-0197338-A1; EP 1310571. See also, WO 2003/042397 (AAV7 and other simian AAV), U.S. Pat. Nos. 7,790,449 and 7,282,199 (AAV8), WO 2005/033321 and U.S. Pat. No. 7,906,111 (AAV9), and WO 2006/110689, WO 2003/042397 (rh.10) and WO 2018/160582 (AAVhu68). These documents also describe other AAV which may be selected for generating AAV and are incorporated by reference. Unless otherwise specified, the AAV capsid, ITRs, and other selected AAV components described herein, may be readily selected from among any AAV, including, without limitation, the AAVs commonly identified as AAV1, AAV2, AAV3, AAV4, AAV5, AAV6, AAV7, AAV8, AAV9, AAV8 bp, AAV7M8, AAVAnc80, AAVrh10, and AAVPHP.B and variants of any of the known or mentioned AAVs or AAVs yet to be discovered or variants or mixtures thereof. See, e.g., WO 2005/033321, which is incorporated herein by reference. In one embodiment, the AAV capsid is an AAV1 capsid or variant thereof, AAV8 capsid or variant thereof, an AAV9 capsid or variant thereof, an AAVrh.10 capsid or variant thereof, an AAVrh64R1 capsid or variant thereof, an AAVhu.37 capsid or variant thereof, or an AAV3B or variant thereof.

The amino acid sequence for the AAVPHP.B variant capsid is publicly available (GenBank Accession No. KU056473) and is as follows:

(SEQ ID NO: 1)
MAADGYLPDWLEDNLSEGIREWWALKPGAPQPKANQQHQDNARGLVLPGY
KYLGPGNGLDKGEPVNAADAAALEHDKAYDQQLKAGDNPYLKYNHADAEF
QERLKEDTSFGGNLGRAVFQAKKRLLEPLGLVEEAAKTAPGKKRPVEQSP
QEPDSSAGIGKSGAQPAKKRLNFGQTGDTESVPDPQPIGEPPAAPSGVGS
LTMASGGGAPVADNNEGADGVGSSSGNWHCDSQWLGDRVITTSTRTWALP
TYNNHLYKQISNSTSGGSSNDNAYFGYSTPWGYFDFNRFHCHFSPRDWQR
LINNNWGFRPKRLNFKLFNIQVKEVTDNNGVKTIANNLTSTVQVFTDSDY
QLPYVLGSAHEGCLPPFPADVFMIPQYGYLTLNDGSQAVGRSSFYCLEYF
PSQMLRTGNNFQFSYEFENVPFHSSYAHSQSLDRLMNPLIDQYLYYLSRT
INGSGQNQQTLKFSVAGPSNMAVQGRNYIPGPSYRQQRVSTTVTQNNNSE
FAWPGASSWALNGRNSLMNPGPAMASHKEGEDRFFPLSGSLIFGKQGTGR
DNVDADKVMITNEEEIKTTNPVATESYGQVATNHQSAQTLAVPFKAQAQT
GWVQNQGILPGMVWQDRDVYLQGPIWAKIPHTDGNFHPSPLMGGFGMKHP
PPQILIKNTPVPADPPTAFNKDKLNSFITQYSTGQVSVEIEWELQKENSK
RWNPEIQYTSNYYKSNNVEFAVNTEGVYSEPRPIGTRYLTRNL

The AAVPHP.eB variant capsid was described by Chan et al. (Nat. Neurosci., Aug. 2017, 20(8):1172-9). The amino acid sequence is publicly available (GenBank Accession No. MF187357.1) and is as follows:

(SEQ ID NO: 2)
MAADGYLPDWLEDNLSEGIREWWALKPGAPQPKANQQHQDNARGLVLPGY
KYLGPGNGLDKGEPVNAADAAALEHDKAYDQQLKAGDNPYLKYNHADAEF
QERLKEDTSFGGNLGRAVFQAKKRLLEPLGLVEEAAKTAPGKKRPVEQSP
QEPDSSAGIGKSGAQPAKKRLNFGQTGDTESVPDPQPIGEPPAAPSGVGS
LTMASGGGAPVADNNEGADGVGSSSGNWHCDSQWLGDRVITTSTRTWALP
TYNNHLYKQISNSTSGGSSNDNAYFGYSTPWGYFDFNRFHCHFSPRDWQR
LINNNWGFRPKRLNFKLFNIQVKEVTDNNGVKTIANNLTSTVQVFTDSDY
QLPYVLGSAHEGCLPPFPADVFMIPQYGYLTLNDGSQAVGRSSFYCLEYF
PSQMLRTGNNFQFSYEFENVPFHSSYAHSQSLDRLMNPLIDQYLYYLSRT
INGSGQNQQTLKFSVAGPSNMAVQGRNYIPGPSYRQQRVSTTVTQNNNSE
FAWPGASSWALNGRNSLMNPGPAMASHKEGEDRFFPLSGSLIFGKQGTGR
DNVDADKVMITNEEEIKTTNPVATESYGQVATNHQSDGTLAVPFKAQAQT
GWVQNQGILPGMVWQDRDVYLQGPIWAKIPHTDGNFHPSPLMGGFGMKHP
PPQILIKNTPVPADPPTAFNKDKLNSFITQYSTGQVSVEIEWELQKENSK
RWNPEIQYTSNYYKSNNVEFAVNTEGVYSEPRPIGTRYLTRNL

The AAV capsid contains 9 hypervariable regions (HVR) which show the most sequence divergence throughout AAV isolates. See, Govindasamy et al, J Virol. 2006 December; 80(23):11556-70. Epub 2006 Sep. 13, which is incorporated herein by reference. The AAV9 VP, for example, differs in nine variable surface regions (VR-I to -IX) compared to AAV4, but at only three (VR-I, VR-II, and VR-IV) compared to AAV2 and AAV8. See, e.g., DiMattia et al. J Virol. 2012 June; 86(12):6947-58, which is incorporated herein by reference. In one embodiment, the AAV capsid has one or more mutations in an HVR such as HVRVIII. In certain embodiments, the mutations in the HVRVIII region confer specific binding for a GPI-anchored protein on the BBB (i.e. forms a binding partner). Thus, an AAV capsid protein can be engineered to specifically bind a GPI-anchored ligand on the BBB to alter its infectivity or to promote delivery of an effector entity conjugated thereto.

In one embodiment, AAV capsids are provided which have been modified or linked to other molecules (for example, an antibody) using methods previously described to generate a binding partner (i.e. the capsid specifically binds a GPI-anchored ligand on the BBB via an intermediate). Thus, the AAV capsid of the conjugates described herein may bind a ligand directly or be conjugated to an intermediate (e.g. an antibody or oligonucleotide), forming a binding partner for the ligand. Methods that can be used to generate an AAV capsid with altered receptor binding and/or to introduce a receptor binding partner are known in the art and provided, for example, in the following references, which are incorporated by reference herein: Münch et al. Molecular Therapy, 2013 January; 21(1):109-18; Ried et al. J Virol. 2002 May; 76(9): 4559-66; Ponnazhagan et al. J Virol. 2002 December; 76(24): 12900-07; and Katrekar D et al. Sci Rep. 2018 Feb. 26; 8(1):3589.

As used herein, a “conjugate” or “conjugated” refers to, for example, an AAV capsid joined to one or more effector entities. In certain embodiments, an AAV capsid and an effector entity are joined by a linker. A “linker” as used herein refers to a chemical linker or a single chain peptide linker that covalently connects the AAV capsid and effector entity of the conjugates described herein. Covalent conjugation can either be direct or via a linker. In certain embodiments, direct conjugation is by formation of a covalent bond between a reactive group on the capsid and a corresponding group or acceptor on the, e.g., neurological drug. In certain embodiments, direct conjugation is by modification (e.g., genetic modification) of one of the two molecules to be conjugated to include a reactive group (as non-limiting examples, a sulfhydryl group or a carboxyl group) that forms a covalent attachment to the other molecule to be conjugated under appropriate conditions. As one non-limiting example, a molecule (e.g., an amino acid) with a desired reactive group (e.g., a cysteine residue) may be introduced into the capsid and a disulfide bond formed with the e.g., neurological drug. Methods for covalent conjugation of nucleic acids to proteins are also known in the art (e.g., photocrosslinking; see, e.g., Zatsepin et al. Russ. Chem. Rev. 74: 77-95 (2005)). Non-covalent conjugation can be any non-covalent attachment means, including hydrophobic bonds, ionic bonds, electrostatic interactions, and the like, as will be readily understood by one of ordinary skill in the art.

Conjugation may also be performed using a variety of linkers. For example, an antibody and a neurological drug may be conjugated using a variety of bifunctional protein coupling agents such as N-succinimidyl-3-(2-pyridyldithio) propionate (SPDP), succinimidyl-4-(N-maleimidom ethyl) cyclohexane-1-carboxylate (SMCC), iminothiolane (IT), bifunctional derivatives of imidoesters (such as dimethyl adipimidate H), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See W094/1 1026. Peptide linkers, comprised of from one to twenty amino acids joined by peptide bonds, may also be used. In certain such embodiments, the amino acids are selected from the twenty naturally-occurring amino acids. In certain other such embodiments, one or more of the amino acids are selected from glycine, alanine, proline, asparagine, glutamine and lysine. The linker may be a “cleavable linker” facilitating release of the neurological drug upon delivery to the brain. For example, an acid-labile linker, peptidase-sensitive linker, photolabile linker, dimethyl linker or disulfide-containing linker (Chari et al., Cancer Res. 52:127-131 (1992); U.S. Pat. No. 5,208,020) may be used.

Conjugates may include, but are not limited to, conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, Ill., U.S.A).

A “CNS antigen” or “CNS target”, as used herein, is an antigen and/or molecule expressed in the CNS, including the brain, which can be targeted with an effector entity, such as an antibody or small molecule. Examples of such antigens and/or molecules include, without limitation: beta-secretase 1 (BACE1), amyloid beta (Abeta), epidermal growth factor receptor (EGFR), human epidermal growth factor receptor 2 (HER2), Tau, apolipoprotein E4 (ApoE4), alpha-synuclein, CD20, huntingtin, prion protein (PrP), leucine rich repeat kinase 2 (LRRK2), parkin, presenilin 1, presenilin 2, gamma secretase, death receptor 6 (DR6), amyloid precursor protein (APP), p75 neurotrophin receptor (p75NTR), and caspase 6.

In certain embodiments, compositions described herein are useful for the treatment of Alzheimer's Disease and include an antibody selected from adumanucab (Biogen), Bapineuzumab (Elan; a humanised mAb directed at the amino terminus of Aβ); Solanezumab Eli Lilly, a humanized mAb against the central part of soluble Aβ); Gantenerumab (Chugai and Hoffmann-La Roche, is a full human mAb directed against both the amino terminus and central portions of Aβ); Crenezumab (Genentech, a humanized mAb that acts on monomeric and conformational epitopes, including oligomeric and protofibrillar forms of Aβ; BAN2401 (Esai Co., Ltd, a humanized immunoglobulin G1 (IgG1) mAb that selectively binds to Aβ protofibrils and is thought to either enhance clearance of Aβ protofibrils and/or to neutralize their toxic effects on neurons in the brain); GSK 933776 (a humanised IgG1 monoclonal antibody directed against the amino terminus of Aβ); AAB-001, AAB-002, AAB-003 (Fc-engineered bapineuzumab); SAR228810 (a humanized mAb directed against protofibrils and low molecular weight Aβ); BIIB037/BART (a full human IgG1 against insoluble fibrillar human Aβ, Biogen Idec), and an anti-Aβ antibody such m266 (relative specificity for Aβ oligomers) [Brody and Holtzman, Annu Rev Neurosci, 2008; 31: 175-193].

In another embodiment, the compositions described herein comprise an useful for the treatment of Parkinson's disease, such as an antibody directed to leucine-rich repeat kinase 2, dardarin (LRRK2), synuclein, alpha-synuclein, or DJ-1 (PARK7). Other antibodies may include, PRX002 (Prothena and Roche) Parkinson's disease and related synucleinopathies. These antibodies, particularly anti-synuclein antibodies may also be useful in the treatment of one or more lysosomal storage disease.

In yet another embodiment, the compositions described herein comprise an antibody that is useful for treating multiple sclerosis, such as natalizumab (a humanized anti-a4-ingrin, iNATA, Tysabri, Biogen Idec and Elan Pharmaceuticals), which was approved in 2006, alemtuzumab (Campath-1H, a humanized anti-CD52), rituximab (rituzin, a chimeric anti-CD20), daclizumab (Zenepax, a humanized anti-CD25), ocrelizumab (humanized, anti-CD20, Roche), ustekinumab (CNTO-1275, a human anti-IL 12 p40+IL23p40); anti-LINGO-1, and ch5D12 (a chimeric anti-CD40), and rHIgM22 (a remyelinated monoclonal antibody; Acorda and the Mayo Foundation for Medical Education and Research). Still other anti-a4-integrin antibodies, anti-CD20 antibodies, anti-CD52 antibodies, anti-IL17, anti-CD19, anti-SEMA4D, and anti-CD40 antibodies may be delivered via the conjugates described herein.

In one embodiment, the compositions provided herein include antibodies useful for the treatment of ALS, such as an antibody against enzyme superoxide dismutase 1 (SOD1) and variants thereof (e.g., ALS variant G93A, C4F6 SOD1 antibody); MS785, which directed to Derlin-1-binding region); and antibodies against neurite outgrowth inhibitor (NOGO-A or Reticulon 4), e.g., GSK1223249, ozanezumab (humanized, GSK, also described as useful for multiple sclerosis).

Methods of generating AAV capsid, coding sequences therefore, and methods for production of rAAV viral vectors have been described. See, e.g., Gao, et al, Proc. Natl. Acad. Sci. U.S.A. 100 (10), 6081-6086 (2003) and US 2015/0315612. In one embodiment, the AAV supplying the capsid is AAV1 or variant thereof. In another embodiment, the AAV supplying the capsid is AAV3B or variant thereof. In one embodiment, the AAV supplying the capsid is AAV9 or variant thereof. In yet another embodiment, the AAV supplying the capsid is a Clade E AAV or variant thereof. Such AAV include rh.2; rh.10; rh.25; bb.1, bb.2, pi.1, pi.2, pi.3, rh.38, rh.40, rh.43, rh.49, rh.50, rh.51, rh.52, rh.53, rh.57, rh.58, rh.61, rh.64, hu.6, hu.17, hu.37, hu.39, hu.40, hu.41, hu.42, hu.66, and hu.67. This clade further includes modified rh.2; modified rh.58; and modified rh.64. See, WO 2005/033321, which is incorporated herein by reference.

As used herein, relating to AAV, the term “variant” means any AAV sequence which is derived from a known AAV sequence, including those sharing at least 70%, at least 75%, at least 80%, at least 85%, at least 90%, at least 95%, at least 97%, at least 99% or greater sequence identity over the amino acid or nucleic acid sequence. In another embodiment, the AAV capsid includes variants which may include up to about 10% variation from any described or known AAV capsid sequence. That is, the AAV capsid shares about 90% identity to about 99.9% identity, about 95% to about 99% identity or about 97% to about 98% identity to an AAV capsid provided herein and/or known in the art. In one embodiment, the AAV capsid shares at least 95% identity with an AAV capsid. When determining the percent identity of an AAV capsid, the comparison may be made over any of the variable proteins (e.g., vp1, vp2, or vp3). In another embodiment, a self-complementary AAV is used.

As used herein, “artificial AAV” means, without limitation, an AAV with a non-naturally occurring capsid protein. Such an artificial capsid may be generated by any suitable technique, using a selected AAV sequence (e.g., a fragment of a vp1 capsid protein) in combination with heterologous sequences which may be obtained from a different selected AAV, non-contiguous portions of the same AAV, from a non-AAV viral source, or from a non-viral source. An artificial AAV may be, without limitation, a pseudotyped AAV, a chimeric AAV capsid, a recombinant AAV capsid, or a “humanized” AAV capsid. Pseudotyped vectors, wherein the vector genome packaged within the AAV capsid has inverted terminal repeats (ITRs) from a source difference from the capsid, are useful in the invention. In one embodiment, AAV2/5 and AAV2/8, which have ITRs from AAV2 and a capsid from AAV5 and AAV8, respectively, are exemplary pseudotyped vectors. The selected genetic element may be delivered by any suitable method, including transfection, electroporation, liposome delivery, membrane fusion techniques, high velocity DNA-coated pellets, viral infection and protoplast fusion. The methods used to make such constructs are known to those with skill in nucleic acid manipulation and include genetic engineering, recombinant engineering, and synthetic techniques. See, e.g., Green and Sambrook, Molecular Cloning: A Laboratory Manual, Cold Spring Harbor Press, Cold Spring Harbor, N.Y. (2012).

In one aspect, the AAV capsid that forms a conjugate with an effector entity is an “empty” capsid. Such empty capsids may contain no detectable genomic sequences of an expression cassette, or only partially packaged genomic sequences which are insufficient to achieve expression of the gene product.

In yet another aspect, the AAV capsids provided include a vector genome having an expression cassette comprising a nucleic acid sequence encoding a heterologous gene. As used herein, a “vector genome” refers to the nucleic acid sequence packaged inside the AAV capsid which forms a viral particle. Such a nucleic acid sequence contains AAV inverted terminal repeat sequences (ITRs). A vector genome contains, at a minimum, from 5′ to 3′, an AAV2 5′ ITR, a heterologous coding sequence, and an AAV2 3′ ITR. However, ITRs from a different source AAV other than AAV2 may be selected. Further, other ITRs may be used. Further, the vector genome preferably contains regulatory sequences which direct expression of the gene of interest. In one aspect, it is preferable to use tissue-specific promoters that are suitable for expression of a heterologous gene or expression cassette delivered to the CNS.

The term “heterologous” as used to describe a nucleic acid sequence or protein means that the nucleic acid or protein was derived from a different organism or a different species of the same organism than the host cell or subject in which it is expressed. The term “heterologous” when used with reference to a protein or a nucleic acid in a plasmid, expression cassette, or vector, indicates that the protein or the nucleic acid is present with another sequence or subsequence with which the protein or nucleic acid in question is not found in the same relationship to each other in nature. Thus, in one aspect, an AAV capsid provided herein comprises a sequence encoding at least one gene which can be therapeutically beneficial, particularly when expressed in the CNS.

An “effector entity” refers to a molecule that is to be transported to the CNS across the BBB. The effector entity typically has a characteristic therapeutic activity that is desired to be delivered to the brain. Effector entities include drugs for treatment of neurological disorders and cytotoxic agents such as e.g. peptides, proteins, nucleic acids (e.g., siRNA), antibodies, in particular monoclonal antibodies or fragments thereof, small molecules, and lipid nanoparticles, which may be directed to a brain target. In certain aspects, an effector entity may comprise a detectable label useful for diagnostic imaging of the CNS.

In one aspect, the AAV capsids are conjugated with a neurological disorder drug, a chemotherapeutic agent and/or an imaging agent in order to transport the drug, chemotherapeutic agent and/or the imaging agent across the BBB.

For a neuropathy disorder, a neurological drug may be selected that is an analgesic including, but not limited to, a narcotic/opioid analgesic (i.e., morphine, fentanyl, hydrocodone, meperidine, methadone, oxymorphone, pentazocine, propoxyphene, tramadol, codeine and oxycodone), a nonsteroidal anti-inflammatory drug (NSAID) (i.e., ibuprofen, naproxen, diclofenac, diflunisal, etodolac, fenoprofen, flurbiprofen, indomethacin, ketorolac, mefenamic acid, meloxicam, nabumetone, oxaprozin, piroxicam, sulindac, and tolmetin), a corticosteroid (i.e., cortisone, prednisone, prednisolone, dexamethasone, methylprednisolone and triamcinolone), an antimigraine agent (i.e., sumatriptin, almotriptan, frovatriptan, sumatriptan, rizatriptan, eletriptan, zolmitriptan, dihydroergotamine, eletriptan and ergotamine), acetaminophen, a salicylate (i.e., aspirin, choline salicylate, magnesium salicylate, diflunisal, and salsalate), an anti-convulsant (i.e., carbamazepine, clonazepam, gabapentin, lamotrigine, pregabalin, tiagabine, and topiramate), an anaesthetic (i.e., isoflurane, trichloroethylene, halothane, sevoflurane, benzocaine, chloroprocaine, cocaine, cyclomethycaine, dimethocaine, propoxycaine, procaine, novocaine, proparacaine, tetracaine, articaine, bupivacaine, carticaine, cinchocaine, etidocaine, levobupivacaine, lidocaine, mepivacaine, piperocaine, prilocaine, ropivacaine, trimecaine, saxitoxin and tetrodotoxin), and a cox-2-inhibitor (i.e., celecoxib, rofecoxib, and valdecoxib). For a neuropathy disorder with vertigo involvement, a neurological drug may be selected that is an anti-vertigo agent including, but not limited to, meclizine, diphenhydramine, promethazine and diazepam. For a neuropathy disorder with nausea involvement, a neurological drug may be selected that is an anti-nausea agent including, but not limited to, promethazine, chlorpromazine, prochlorperazine, trimethobenzamide, and metoclopramide. For a neurodegenerative disease, a neurological drug may be selected that is a growth hormone or neurotrophic factor; examples include but are not limited to brain-derived neurotrophic factor (BDNF), nerve growth factor (NGF), neurotrophin-4/5, fibroblast growth factor (FGF)-2 and other FGFs, neurotrophin (NT)-3, erythropoietin (EPO), hepatocyte growth factor (HGF), epidermal growth factor (EGF), transforming growth factor (TGF)-alpha, TGF-beta, vascular endothelial growth factor (VEGF), interleukin-1 receptor antagonist (IL-Ira), ciliary neurotrophic factor (CNTF), glial-derived neurotrophic factor (GDNF), neurturin, platelet-derived growth factor (PDGF), heregulin, neuregulin, artemin, persephin, interleukins, glial cell line derived neurotrophic factor (GFR), granulocyte-colony stimulating factor (CSF), granulocyte-macrophage-CSF, netrins, cardiotrophin-1, hedgehogs, leukemia inhibitory factor (LIF), midkine, pleiotrophin, bone morphogenetic proteins (BMPs), netrins, saposins, semaphorins, and stem cell factor (SCF).

For cancer, a neurological drug may be a chemotherapeutic agent. Examples of chemotherapeutic agents include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphor-amide and trimethylolomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-1 1 (irinotecan, CAMPTOSAR®), acetylcamptothecin, scopolectin, and 9-amino camptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamme, mechlorethamme oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (see, e.g., Agnew, Chem Intl. Ed. Engl, 33: 183-186 (1994)); dynemicin, including dynemicin A; an esperamicin; as well as neocarzino statin chromophore and related chromoprotein enediyne antibiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; eflornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoids, e.g., TAXOL® paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTEPvE® doxetaxel (Rhone-Poulenc Rorer, Antony, France); chloranbucil; gemcitabine (GEMZAR®); 6-thioguanine; mercaptopurine; methotrexate; platinum analogs such as cisplatin and carboplatin; vinblastine (VELBAN®); platinum; etoposide (VP-16); ifosfamide; mitoxantrone; vincristine (ONCOVIN®); oxaliplatin; leucovovin; vinorelbine (NAVELBINE®); novantrone; edatrexate; daunomycin; aminopterin; ibandronate; topoisomerase inhibitor RFS 2000; difluorometlhylornithine (DMFO); retinoids such as retinoic acid; capecitabine (XELODA®); pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovovin.

Also useful in certain aspects of the invention described herein are chemotherapeutic agents that are anti-hormonal agents that act to regulate, reduce, block, or inhibit the effects of hormones that can promote the growth of cancer, and are often in the form of systemic, or whole-body treatment. They may be hormones themselves. Examples include anti-estrogens and selective estrogen receptor modulators (SERMs), including, for example, tamoxifen (including NOLVADEX® tamoxifen), EVISTA® raloxifene, droloxifene, 4-hydroxytamoxifen, trioxifene, keoxifene, LY 117018, onapristone, and FARESTON® toremifene; anti-progesterones; estrogen receptor down-regulators (ERDs); agents that function to suppress or shut down the ovaries, for example, leutinizing hormonereleasing hormone (LHRH) agonists such as LUPRON® and ELIGARD® leuprolide acetate, goserelin acetate, buserelin acetate and tripterelin; other anti-androgens such as flutamide, nilutamide and bicalutamide; and aromatase inhibitors that inhibit the enzyme aromatase, which regulates estrogen production in the adrenal glands, such as, for example, 4(5)-imidazoles, amino glutethimide, MEGASE® megestrol acetate, AROMASIN® exemestane, formestanie, fadrozole, RIVISOR® vorozole, FEMARA® letrozole, and ARIMIDEX® anastrozole. In addition, such definition of chemotherapeutic agents includes bisphosphonates such as clodronate (for example, BONEFOS® or OSTAC®), DIDROCAL® etidronate, NE-58095, ZOMETA® zoledronic acid/zoledronate, FOSAMAX® alendronate, AREDIA® pamidronate, SKELID® tiludronate, or ACTONEL® risedronate; as well as troxacitabine (a 1,3-dioxolane nucleoside cytosine analog); antisense oligonucleotides, particularly those that inhibit expression of genes in signaling path ways implicated in aberrant cell proliferation, such as, for example, PKC-alpha, Raf, H-Ras, and epidermal growth factor receptor (EGF-R); vaccines such as THERATOPE® vaccine and gene therapy vaccines, for example, ALLOVECTIN® vaccine, LEUVECTIN® vaccine, and VAXID® vaccine; LURTOTECAN® topoisomerase 1 inhibitor; ABARELLX® rmRH; lapatinib ditosylate (an ErbB-2 and EGFR dual tyrosine kinase small-molecule inhibitor also known as GW572016); and pharmaceutically acceptable salts, acids or derivatives of any of the above. Another group of compounds that may be selected as neurological drugs for cancer treatment or prevention are anti-cancer immunoglobulins (including, but not limited to, trastuzumab, bevacizumab, alemtuxumab, cetuximab, gemtuzumab ozogamicin, ibritumomab tiuxetan, panitumumab and rituximab). In some instances, antibodies in conjunction with a toxic label may be used to target and kill desired cells (i.e., cancer cells), including, but not limited to, tositumomab with a radio label.

For a lysosomal storage disease, a neurological drug may be selected that is itself or otherwise mimics the activity of the enzyme that is impaired in the disease. Exemplary recombinant enzymes for the treatment of lysosomal storage disorders include, but are not limited to those set forth in e.g., U.S. Patent Application publication no. 2005/0142141 (i.e., alpha-L-iduronidase, iduronate-2-sulphatase, N-sulfatase, alpha-N-acetylglucosaminidase, N-acetyl-galactosamine-6-sulfatase, beta-galactosidase, arylsulphatase B, beta-glucuronidase, acid alpha-glucosidase, glucocerebrosidase, alpha-galactosidase A, hexosaminidase A, acid sphingomyelinase, betagalactocerebrosidase, beta-galactosidase, arylsulfatase A, acid ceramidase, aspartoacylase, palmitoyl-protein thioesterase 1 and trip eptidyl amino peptidase 1).

For amyloidosis, a neurological drug may be selected that includes, but is not limited to, an antibody or other binding molecule (including, but not limited to a small molecule, a peptide, an aptamer, or other protein binder) that specifically binds to a target selected from: beta secretase, tau, presenilin, amyloid precursor protein or portions thereof, amyloid beta peptide or oligomers or fibrils thereof, death receptor 6 (DR6), receptor for advanced glycation endproducts (RAGE), parkin, and huntingtin; a cholinesterase inhibitor (i.e., galantamine, donepezil, rivastigmine and tacrine); an NMD A receptor antagonist (i.e., memantine), a monoamine depletory (i.e., tetrabenazine); an ergoloid mesylate; an anticholinergic antiparkinsonism agent (i.e., procyclidine, diphenhydramine, trihexylphenidyl, benztropine, biperiden and trihexyphenidyl); a dopaminergic antiparkinsonism agent (i.e., entacapone, selegiline, pramipexole, bromocriptine, rotigotine, selegiline, ropinirole, rasagiline, apomorphine, carbidopa, levodopa, pergolide, tolcapone and amantadine); a tetrabenazine; an anti-inflammatory (including, but not limited to, a nonsteroidal anti-inflammatory drug (i.e., indomethicin and other compounds listed above); a hormone (i.e., estrogen, progesterone and leuprolide); a vitamin (i.e., folate and nicotinamide); a dimebolin; a homotaurine (i.e., 3-aminopropanesulfonic acid; 3 APS); a serotonin receptor activity modulator (i.e., xaliproden); an interferon, and a glucocorticoid.

For a viral or microbial disease, a neurological drug may be selected that includes, but is not limited to, an antiviral compound (including, but not limited to, an adamantane antiviral (i.e., rimantadine and amantadine), an antiviral interferon (i.e., peginterferon alfa-2b), a chemokine receptor antagonist (i.e., maraviroc), an integrase strand transfer inhibitor (i.e., raltegravir), a neuraminidase inhibitor (i.e., oseltamivir and zanamivir), a non-nucleoside reverse transcriptase inhibitor (i.e., efavirenz, etravirine, delavirdine and nevirapine), a nucleoside reverse transcriptase inhibitor (tenofovir, abacavir, lamivudine, zidovudine, stavudine, entecavir, emtricitabine, adefovir, zalcitabine, telbivudine and didanosine), a protease inhibitor (i.e., darunavir, atazanavir, fosamprenavir, tipranavir, ritonavir, nelfmavir, amprenavir, indinavir and saquinavir), a purine nucleoside (i.e., valacyclovir, famciclovir, acyclovir, ribavirin, ganciclovir, valganciclovir and cidofovir), and a miscellaneous antiviral (i.e., enfuvirtide, foscarnet, palivizumab and fomivirsen)), an antibiotic (including, but not limited to, an aminopenicillin (i.e., amoxicillin, ampicillin, oxacillin, nafcillin, cloxacillin, dicloxacillin, flucoxacillin, temocillin, azlocillin, carbenicillin, ticarcillin, mezlocillin, piperacillin and bacampicillin), a cephalosporin (i.e., cefazolin, cephalexin, cephalothin, cefamandole, ceftriaxone, cefotaxime, cefpodoxime, ceftazidime, cefadroxil, cephradine, loracarbef, cefotetan, cefuroxime, cefprozil, cefaclor, and cefoxitin), a carbapenem/penem (i.e., imipenem, meropenem, ertapenem, faropenem and doripenem), a monobactam (i.e., aztreonam, tigemonam, norcardicin A and tabtoxinine-beta-lactam, a betalactamase inhibitor (i.e., clavulanic acid, tazobactam and sulbactam) in conjunction with another beta-lactam antibiotic, an aminoglycoside (i.e., amikacin, gentamicin, kanamycin, neomycin, netilmicin, streptomycin, tobramycin, and paromomycin), an ansamycin (i.e., geldanamycin and herbimycin), a carbacephem (i.e., loracarbef), a glycopeptides (i.e., teicoplanin and vancomycin), a macrolide (i.e., azithromycin, clarithromycin, dirithromycin, erythromycin, roxithromycin, troleandomycin, telithromycin and spectinomycin), a monobactam (i.e., aztreonam), a quinolone (i.e., ciprofloxacin, enoxacin, gatifloxacin, levofloxacin, lomefloxacin, moxifloxacin, nor floxacin, ofloxacin, trovafloxacin, grepafloxacin, sparfloxacin and temafloxacin), a sulfonamide (i.e., mafenide, sulfonamidochrysoidme, sulfacetamide, sulfadiazine, sulfamethizole, sulfanilamide, sulfasalazine, sulfisoxazole, trimethoprim, trimethoprim and sulfamethoxazole), a tetracycline (i.e., tetracycline, demeclocycline, doxycycline, minocycline and oxytetracycline), an antineoplastic or cytotoxic antibiotic (i.e., doxorubicin, mitoxantrone, bleomycin, daunorubicin, dactinomycin, epirubicin, idarubicin, plicamycin, mitomycin, pentostatin and valrubicin) and a miscellaneous antibacterial compound (i.e., bacitracin, colistin and polymyxin B)), an antifungal (i.e., metronidazole, nitazoxanide, imidazole, chloroquine, iodoquinol and paromomycin), and an antiparasitic (including, but not limited to, quinine, chloroquine, amodiaquine, pyrimethamine, sulphadoxine, proguanil, mefloquine, atovaquone, primaquine, artemesinin, halofantrine, doxycycline, clindamycin, mebendazole, pyrantel pamoate, thiabendazole, diethylcarbamazine, ivermectin, rifampin, amphotericin B, melarsoprol, efornithine and albendazole). For ischemia, a neurological drug may be selected that includes, but is not limited to, a thrombolytic (i.e., urokinase, alteplase, reteplase and tenecteplase), a platelet aggregation inhibitor (i.e., aspirin, cilostazol, clopidogrel, prasugrel and dipyridamole), a statin (i.e., lovastatin, pravastatin, fiuvastatin, rosuvastatin, atorvastatin, simvastatin, cerivastatin and pitavastatin), and a compound to improve blood flow or vascular flexibility, including, e.g., blood pressure medications.

For a behavioral disorder, a neurological drug may be selected from a behavior-modifying compound including, but not limited to, an atypical antipsychotic (i.e., risperidone, olanzapine, apripiprazole, quetiapine, paliperidone, asenapine, clozapine, iloperidone and ziprasidone), a phenothiazine antipsychotic (i.e., prochlorperazine, chlorpromazine, fluphenazine, perphenazine, trifluoperazine, thioridazine and mesoridazine), a thioxanthene (i.e., thiothixene), a miscellaneous antipsychotic (i.e., pimozide, lithium, molindone, haloperidol and loxapine), a selective serotonin reuptake inhibitor (i.e., citalopram, escitalopram, paroxetine, fluoxetine and sertraline), a serotonin-norepinephrine reuptake inhibitor (i.e., duloxetine, venlafaxine, desvenlafaxine, a tricyclic antidepressant (i.e., doxepin, clomipramine, amoxapine, nortriptyline, amitriptyline, trimipramine, imipramine, protriptyline and desipramine), a tetracyclic antidepressant (i.e., mirtazapine and maprotiline), a phenylpiperazine antidepressant (i.e., trazodone and nefazodone), a monoamine oxidase inhibitor (i.e., isocarboxazid, phenelzine, selegiline and tranylcypromine), a benzodiazepine (i.e., alprazolam, estazolam, flurazeptam, clonazepam, lorazepam and diazepam), a norepinephrine-dopamine reuptake inhibitor (i.e., bupropion), a CNS stimulant (i.e., phentermine, diethylpropion, methamphetamine, dextroamphetamine, amphetamine, methylphenidate, dexmethylphenidate, lisdexamfetamine, modafmil, pemoline, phendimetrazme, benzphetamine, phendimetrazme, armodafmil, diethylpropion, caffeine, atomoxetine, doxapram, and mazindol), an anxiolytic/sedative/hypnotic (including, but not limited to, a barbiturate (i.e., secobarbital, phenobarbital and mephobarbital), a benzodiazepine (as described above), and a miscellaneous anxiolytic/sedative/hypnotic (i.e. diphenhydramine, sodium oxybate, zaleplon, hydroxyzine, chloral hydrate, aolpidem, buspirone, doxepin, eszopiclone, ramelteon, meprobamate and ethclorvynol)), a secretin (see, e.g., Ratliff-Schaub et al. Autism 9: 256-265 (2005)), an opioid peptide (see, e.g., Cowen et al, J. Neurochem. 89:273-285 (2004)), and a neuropeptide (see, e.g., Hethwa et al. Am. J. Physiol. 289: E301-305 (2005)).

For CNS inflammation, a neurological drug may be selected that addresses the inflammation itself (i.e., a non-steroidal anti-inflammatory agent such as ibuprofen or naproxen), or one which treats the underlieing cause of the inflammation (i.e., an anti-viral or anti-cancer agent).

Also provided herein are pharmaceutical compositions comprising the AAV capsid and effector entity conjugates. The pharmaceutical compositions described herein are designed for delivery to subjects in need thereof by any suitable route or a combination of different routes. In certain embodiments, the compositions described herein are administered systemically (e.g., intravenously). In one embodiment, direct delivery to the CNS is desired and may be performed via intrathecal injection. The term “intrathecal administration” refers to delivery that targets the cerebrospinal fluid (CSF). This may be done by direct injection into the ventricular or lumbar CSF, by suboccipital puncture, or by other suitable means. Meyer et al, Molecular Therapy (31 Oct. 2014), demonstrated the efficacy of direct CSF injection which resulted in widespread transgene expression throughout the spinal cord in mice and nonhuman primates when using a 10 times lower dose compared to the IV application. This document is incorporated herein by reference. In one embodiment, the composition is delivered via intracerebroventricular viral injection (see, e.g., Kim et al, J Vis Exp. 2014 Sep. 15; (91):51863, which is incorporated herein by reference). See also, Passini et al, Hum Gene Ther. 2014 July; 25(7):619-30, which is incorporated herein by reference. In another embodiment, the composition is delivered via lumbar injection.

Alternatively, other routes of administration may be selected (e.g., oral, inhalation, intranasal, intratracheal, intraarterial, intraocular, intravenous, intramuscular, intraperitoneal, and other parental routes). Accordingly, pharmaceutical compositions may be formulated for any appropriate route of administration, for example, in the form of liquid solutions or suspensions (as, for example, for intravenous administration, for oral administration, etc.). Alternatively, pharmaceutical compositions may be in solid form (e.g., in the form of tablets or capsules, for example for oral administration). In some embodiments, pharmaceutical compositions may be in the form of powders, drops, aerosols, etc.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

Methods and agents well known in the art for making formulations are described, for example, in “Remington's Pharmaceutical Sciences,” Mack Publishing Company, Easton, Pa. Formulations may, for example, contain excipients, carriers, stabilizers, or diluents such as sterile water, saline, polyalkylene glycols such as polyethylene glycol, oils of vegetable origin, or hydrogenated napthalenes, preservatives (such as octadecyldimethylbenzyl, ammonium chloride, hexamethonium chloride, benzalkonium chloride, benzethonium chloride, phenol, butyl or benzyl alcohol, alkyl parabens such as methyl or propyl paraben, catechol, resorcinol, cyclohexanol, 3-pentanol, and m-cresol), low molecular weight polypeptides, proteins such as serum albumin, gelatin, or immunoglobulins, hydrophilic polymers such as polyvinylpyrrolidone, amino acids such as glycine, glutamine, asparagine, histidine, arginine, and lysine, monosaccharides, disaccharides, and other carbohydrates including glucose, mannose, and dextrins, chelating agents such as EDTA, sugars such as sucrose, mannitol, trehalose or sorbitol; salt-forming counter-ions such as sodium; metal complexes (e.g. Zn-protein complexes); and/or non-ionic surfactants such as TWEEN™ PLURONICS™ or polyethylene glycol (PEG).

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nanoparticles and nanocapsules) or in macroemulsions. Such techniques are disclosed in Remington's Pharmaceutical Sciences 16th edition, Osol, A. Ed. (1980).

The AAV capsid and effector entity conjugates described herein may be utilized in a variety of in vivo methods. In one embodiment, a method of treatment for a neurological disease comprising administering a conjugate described herein is provided. Accordingly, binding of the AAV capsid to a GPI-anchored BBB ligand promotes delivery of an effector entity conjugated thereto to the CNS, wherein the BBB ligand acts as receptor or co-receptor to mediate viral entry or transport of the conjugate across the BBB.

By “administering” or “route of administration” is meant delivery of composition described herein, with or without a pharmaceutical carrier or excipient, to the subject. Routes of administration may be combined, if desired. In some embodiments, the administration is repeated periodically. In certain aspects, compositions described herein are co-administered. In certain aspects, an AVV vector or conjugate of the invention is co-administered with an antibody. Where compositions are co-administered, they may be formulated together or separately to be delivered to the subject essentially simultaneously by the same or different route. In certain aspects, the time period between administering compositions to a subject may be shorter than 1 hour, 2 hours, 4 hours, 8 hours, 12 hours, 24 hours, or 48 hours. In certain embodiments, the compositions described herein are administered to a subject in need thereof one or more times. In certain embodiments, one, two, three or more re-administrations are permitted of a composition. In certain aspects, where a composition comprising an AAV capsid is administered, such re-administration may be with the same type of vector, or a different vector.

In certain embodiments, the AAV capsid of the conjugates provided herein comprises an expression cassette such that delivery of the vector to the CNS results in expression of a heterologous gene. Accordingly, delivery of the AAV capsid to the CNS results in ectopic or altered expression of a gene product to benefit a patient with a neurological disorder.

By “change in expression” or “altered expression” or similar phrases is meant an upregulation in the expression level of a nucleic acid sequence, e.g., genes or transcript, or cell surface marker, in comparison to the selected reference standard or control; a downregulation in the expression level of a nucleic acid sequence, e.g., genes or transcript, or cell surface maker, in comparison to the selected reference standard or control; or a combination of a pattern or relative pattern of certain upregulated and/or down regulated genes and/or cell surface markers. The degree of change in expression can vary with each individual gene or marker, or for among subjects. Thus, in certain aspects, delivery of the vectors or effector entities described herein across the BBB results in a change in expression of a CNS target (i.e., gene, protein, etc.).

In certain embodiments, the methods described herein are useful to the treatment of a lysosomal storage disorders. Lysosomal storage disorders are metabolic disorders which are in some cases associated with the CNS or have CNS-specific symptoms; such disorders include, but are not limited to: Tay-Sachs disease, Gaucher's disease, Fabry disease, mucopolysaccharidosis (types I, II, III, IV, V, VI and VII), glycogen storage disease, GM1-gangliosidosis, metachromatic leukodystrophy, Farber's disease, Canavan's leukodystrophy, and neuronal ceroid lipofuscinoses types 1 and 2, Niemann-Pick Disease Types A and B (acid sphingomyelinase deficiency or ASMD), Niemann-Pick Disease Type C (NPC), Pompe disease, and Krabbe's disease.

In another embodiment, a method altering the infectivity of the CNS by an AAV vector is provided. In one aspect, a co-therapy is provided which comprises co-administering an AAV capsid with a binding partner for a GPI-anchored BBB ligand and an antibody or antibody fragment that binds the BBB ligand. Thus, binding of the antibody or antibody fragment to the GPI-anchored ligand reduces or inhibits viral entry at the BBB. A reduction or inhibition of CNS infectivity can be measured relative to the incidence observed in the absence of the co-administered antibody or antibody fragment. In certain aspects, infectivity in the presence of the antibody may be reduced about 10%, 20%, 30%, 40%, 50%, 60%, 70%, 80%, 90%, 95% or greater relative to infectivity in the absence of the antibody.

Also provided herein is a method of engineering an AAV capsid which is particularly useful targeting the CNS. The engineered capsid comprises peptide fragments (i.e. binding partners) which specifically bind a GPI-anchored protein on the BBB. Accordingly, AAV capsids can be engineered to confer binding or enhance binding to a GPI-anchored protein selected from Ly6E, GRA3, ALPL, BST2, EFNA5, NT5E, DPEP2, GPC1, LYPD5, GPC6, CD14, CA4, GPC5, CD59, TFPI, EFNA1, EFNA3, HYAL2, MELTF, ULBP2, EFNA4, CNTN5, BCAN, RECK, CFC1, SEMA7A, PRNP, LY6E, PRND, PLAUR, CD24A, MMP25, ART3, LYPD1, PIBF1, CAPRIN1, GFRA3, GPIHBP1, MACF1, and SEC24B. In one aspect, the method comprises identifying a peptide fragment that specifically binds a GPI-anchored protein and the amino acid sequence encoding such binding partner. An AVV capsid is then modified to express the binding partner thereby generating an AVV capsid capable of binding or with enhanced binding to a GPI-anchored ligand on the BBB. In one aspect, the method comprises identifying a binding partner for a ligand selected from Ly6E, GRA3, ALPL, BST2, EFNA5, NT5E, DPEP2, GPC1, LYPD5, GPC6, CD14, CA4, GPC5, CD59, TFPI, EFNA1, EFNA3, HYAL2, MELTF, ULBP2, EFNA4, CNTN5, BCAN, RECK, CFC1, SEMA7A, PRNP, LY6E, PRND, PLAUR, CD24A, MMP25, ART3, LYPD1, PIBF1, CAPRIN1, GFRA3, GPIHBP1, MACF1, and SEC24B. In another aspect, the method comprises modifying an AAV HVRVIII site or another HVR site to express a binding partner for a GPI-anchored ligand on the BBB. In yet another aspect, the AAV is selected from AAV1, AAV3B, or AAV9 and the engineered capsid specifically binds a GPI-anchored protein on the BBB. An engineered AAV capsid obtained by these methods can be used for convention gene therapy (i.e. delivery of an expression cassette) and/or conjugated to an effector entity as described herein.

In another embodiment, the conjugates described herein are utilized in methods for delivering a detectable effector entity to the CNS of a subject. In one aspect, delivery of the detectable entity allows a CNS target to be quantified to diagnose a subject with a neurological disorder. In one aspect, the detectable agent binds a CNS target and comprises one or more of a peptide, nucleic acid, siRNA, antibody, antibody fragment, small molecule, lipid nanoparticle, or cytotoxic agent. Accordingly, in certain aspects, the methods provided herein comprise administering a detectable agent and imaging of the CNS using, for example, magnetic resonance imaging (MRI) or computed tomography (CT).

These examples are provided for the purpose of illustration only. The compositions, experimental protocols and methods disclosed and/or claimed herein can be made and executed without undue experimentation in light of the present disclosure. The protocols and methods described in the examples are not considered to be limitations on the scope of the claimed invention. Rather this specification should be construed to encompass any and all variations that become evident as a result of the teaching provided herein. One of skill in the art will understand that changes or variations can be made in the disclosed embodiments of the examples and expected similar results can be obtained. For example, the substitutions of reagents that are chemically or physiologically related for the reagents described herein are anticipated to produce the same or similar results. All such similar substitutes and modifications are apparent to those skilled in the art and fall within the scope of the invention.

### EXAMPLES

### Example 1: GPI-Linked Protein LY6A (SCA-1)-Mediated Transport Across the Blood Brain Barrier

The factors allowing AAV-PHP.B to cross the blood brain barrier (BBB) with such efficiency were not previously defined. In this study, it was determined that the high BBB permeability of AAV-PHP.B is specific to C57BL/6J mice and is based on the specific binding of the 7 amino acid insert to a GPI-anchored protein expressed on brain endothelial cells called LY6A (also known as SCA-1), which has previously been studied in the context of hematopoietic, mesenchymal, and cancer stem cell biology⁹⁻¹².

### Methods

### Animals

All animal protocols were approved by the Institutional Animal Care and Use Committee of the University of Pennsylvania and animals were housed in an AAALAC-accredited barrier facility within the School of Medicine at the University of Pennsylvania. The University of Pennsylvania's Office of Laboratory Animal Welfare (OLAW) Assurance Number is A3079-01. C56BL/6J (stock #000664), BALB/cJ (#000651), and F1 hybrids CB6F1/J (#100007) were purchased from the Jackson Laboratory. F2 hybrids were obtained by crossing CB6F1/J at the facility. Ly6a-null mice on a C57BL/6J and BALB/cJ background were generously provided with William L. Stanford (University of Ottawa). For reporter gene experiments, adult (six to eight weeks old) males were injected. Animals were housed in standard caging of 2 to 5 animals per cage. Cages, water bottles, and bedding substrates were autoclaved into the barrier facility and cages were changed once a week. An automatically controlled 12-hour light/dark cycle was maintained. Each dark period began at 1900 hours (±30 minutes). Irradiated laboratory rodent food was provided ad libitum.

### Cell Lines

Human embryonic Kidney 293 cells (HEK 293, originally obtained from a female embryo) were maintained in Dulbecco's Modified Eagle Medium (DMEM Gibco, ThermoFisher Scientific cat #11995-040) supplemented with gamma-irradiated 10% fetal bovine serum (Hyclone™ cat #SH30071.031R) and 100 IU/mL penicillin/streptomycin, and grown in a humidified incubator at 37° C. with 5% CO₂.

### Vector Production

The AAV9.PHP.B trans plasmid (pAAV2/PHP.B) was generated with QuikChange Lightning Site-Directed Mutagenesis Kit (Agilent Technologies, cat #210515) and pAAV2/9 (Penn Vector Core) as the template, following the manufacturer's manual. The AAV-PHP.B mutants were constructed the same way with pAAV2/PHP.B as the template. AAV vectors were produced and titrated by Penn Vector Core as described before³⁷. Briefly, HEK 293 cells were triple-transfected and the culture supernatant was harvested, concentrated and purified with iodixanol-gradient. The purified vectors were titrated with droplet digital PCR using primers targeting the rabbit Beta-globin polyA sequence as previously described³⁸.

### In Vivo Studies and Histology

Mice received 1×10¹² GC (5×10¹³ GC/kg) of AAV9 or AAV-PHP.B or AAV-PHP.eB vectors encoding enhanced GFP (Penn Vector Core) in 0.1 mL via the lateral tail vein and were euthanized by inhalation of CO2 21 days post injection. Tissues were promptly collected, starting with brain. Half sagittally-sectioned brain was immersion-fixed in 10% neutral buffered formalin for about 24 h, washed briefly in PBS, and equilibrated sequentially in 15% and 30% sucrose in PBS at 4° C. Tissues were then frozen in OCT embedding medium and cryosectioned for direct GFP visualization (brain were sectioned at 30 μm, other tissues at 10 μm thickness). Images were either acquired with a Nikon Eclipse Ti-E fluorescence microscope or whole brain sections were scanned with an Aperio Versa slide scanner. The other half of the brain was either snap-frozen on dry ice for qPCR vector biodistribution study, or formalin-fixed and paraffin-embedded for immunostaining. Immunofluorescence for LY6A was performed on formalin-fixed paraffin-embedded brain samples. Sections were deparaffinized, boiled for 6 min in 10 mM citrate buffer (pH 6.0) for antigen retrieval, and then blocked with 1% donkey serum in PBS+0.2% Triton for 15 min followed by sequential incubation with primary (1 h) and fluorescence-labeled secondary antibodies (45 min) diluted in blocking buffer. Monoclonal rat antibody D7 against LY6A (eBioscience, ThermoFisher Scientific Cat #14-5981-82) was used at a dilution of 1:200 and TRITC-labeled donkey anti-rat (Jackson Immunoresearch cat #712-025-153; 1:100 dilution) served as secondary antibody.

### Vector Biodistribution

Tissue DNA was extracted with QIAamp DNA Mini Kit (Qiagen cat #51306) and vector genomes were quantified by real-time PCR using Taqman reagents (Applied Biosystems, Life Technologies) and primer/probe targeting the rBG polyadenylation sequence of the vectors.

### Whole Exome Sequencing

Whole exome sequencing libraries were generated from genomic DNA isolated from the brains of 4 F1 and 12 F2 mice using the Agilent SureSelect Mouse All Exon Kit (Agilent cat #5190-4641). Samples were indexed and sequenced on a NextSeq high output cartridge (8 samples per flow-cell). Following sequencing, paired-end reads from each sample were mapped to the reference genome (GRCm38) using NovoAlign (v3.08.02) with optimized parameters for whole exome sequencing³⁹. Duplicate reads (optical and/or PCR duplicates that originate from a single fragment of DNA) were subsequently flagged with Picard tools (v2.13.2). Following data pre-processing, variant calling was performed for each sample using GATK best practices⁴⁰⁻⁴³. In brief, base quality score recalibration was first performed, followed by variant calling, and joint genotyping using GATK (v3.8). Raw variants were subsequently filtered to remove Mendelian violations and variants with a QUAL score of 50 or less. High confidence variants were subsequently used for association testing (see statistical analysis). Genomic variant annotation and functional effect prediction was performed using SnpEff⁴⁴.

### Ly6a Expression Vector Construction

LY6A from C57BL/6J and BALB/cJ coding sequences were synthesized as GeneArt Strings (Thermo Fisher Scientific). For native LY6A expression vectors, GeneArt Strings were assembled into BamHI digested pcDNA3.1(+)IRES GFP (Addgene 51406) using NEBuilder HiFi DNA Assembly Master Mix (NEB cat #E2621). For the Twin-Strep-tagged LY6A expression vectors, DNA encoding the first 111 amino acid residues of LY6A were PCR amplified before assembly into Esp3I digested pESG-IBA103 using NEBuilder HiFi DNA Assembly Master Mix (NEB cat #E2621). All constructs were confirmed by Sanger sequencing.

### Twin-Strep-Tagged LY6A Production and Purification

HEK 293 cells were transiently transfected with plasmids expressing Twin-Strep-tagged LY6A (C57BL/6J or BALB/cJ variants) using a 1:2 DNA to polyethylenimine (PEI-linear polyethylenimine hydrochloride MW 40,000, Polysciences, cat #24765-1) w/w ratio. 72 hours post-transfection, cell culture supernatants were filtered through 0.22 μm filters, and adjusted to pH 8.0 by adding one tenth volumes 10× Buffer W (1M Tris-HCl, pH 8.0, 1.5 M NaCl, 10 mM EDTA). Transfected 293 cells were lysed in 1× Buffer W supplemented with 0.1% Triton X-100, and passaged twice through 27-gauge needles. Cell lysates and culture supernatants were combined followed by biotin depletion through a 15-minute incubation with 1/400 volumes BioLock Biotin Blocking Solution (IBA Life Sciences cat #2-0205-050) and a subsequent centrifugation. Purification of Twin-Strep-tagged LY6A proteins was achieved through Strep-Tactin XT affinity chromatography according to the manufacturer's protocol (IBA Life Sciences). Briefly, Strep-Tactin XT Superflow resin (cat #2-4010-010) was incubated with cell lysates and supernatant for two hours at room temperature, washed with four column volumes (CV) of 1× Buffer W, and eluted with 0.6 CV, 1.6 CV and 0.8 CV of 1× Buffer BXT. Eluate fractions containing recombinant LY6A were pooled and dialyzed four times in 50 mM Tris-HCl, pH 8.0, 150 mM NaCl. Protein concentrations were determined by BCA assay (Pierce, cat #23225).

### Enzyme-Linked Immunosorbent Assay (ELISA)

Strep-Tactin XT coated microplates (IBA Life Sciences, cat #2-4101-001) were incubated overnight at 4° C. with 200 μL of coating buffer (50 mM Tris-HCl, pH 8.0, 150 mM NaCl) containing zero or 0.5 μg of Twin-Strep-tagged LY6A proteins per well. Plates were washed 3× with PBST (0.05% Tween-20 in PBS), and blocked in 3% BSA in PBS for two hours at room temperature. AAV serotypes were diluted in PBS supplemented with 1% BSA and 0.1% pluronic F-68 to the indicated concentrations. 200 μL of AAV dilutions were added to each well and incubated for two hours at 37° C. Immobilized AAV particles were detected by sequential one-hour incubations with a rabbit antiserum against AAV9 (1:50,000, Penn Immunology Core) and a horseradish peroxidase (HRP)-conjugated goat anti-rabbit secondary antibody (1:5,000, Thermo Fisher Scientific, ct #31460). Plates were developed using 200 μL of the SureBlue TMB 1-component microwell Peroxidase Substrate (Seracare, cat #52-00-01) per product instructions, and optical densities (OD) measured at 450 nm by microplate reader (SpectraMax M3). To calculate LY6A-dependent AAV binding, background AAV bindings to uncoated microplate wells were subtracted from the observed AAV bindings to LY6A-coated wells at each virus concentrations. Data are representative of three independent experiments. To estimate the apparent affinity (Kd) of AAV-PHP.B variants for LY6A variants, we applied least squares fitting of the ELISA data (A450 signal as a fraction of the maximum) to a simple 1:1 model for molecular interaction [f=At/(Kd+At)], where f is the fraction of Ly6a bound, and At is the total amount of AAV applied to the well. A more complex model that accounted for Ly6a explicitly did not improve the fit. The ELISA orientation used allows us only to estimate the apparent affinity of a vector for a field of LY6A protein, as might be encountered on a cell, and does not represent the microscopic affinity of a single PHP.B peptide loop with a single LY6A protein.

### HEK293 Transduction Assay

All cell culture was performed in a humidified incubator at 37° C.+5% CO¬2 using DMEM medium+10% Heat Inactivated Fetal Bovine Serum (FBS)+1% Penicillin Streptomycin (P/S). On the first day of the assay, HEK293 cells were trypsinized, counted, and seeded in 96 well plates (Corning #3603) at a density of 80,000 cells/well. 20-24 h later plasmid constructs containing the C57BL/6J or Balb/cJ alleles of Ly6a or Ly6c1, upstream of an IRES2-EGFP sequence, were transiently transfected into cells. Transfection was performed in serum-free DMEM using 0.14 ug plasmid DNA and 0.28 ug PEI per well in a culture volume of 100 uL. Mock transfected cells received only PEI in serum-free DMEM. 24 h after transfection, each well was supplemented with 100 uL DMEM+20% FBS+1% P/S and allowed to grow out under full serum conditions for an additional 24 h. 48 h after transfection, GFP expression was assessed qualitatively in transfected wells using a fluorescence microscope. AAV9 and AAV9-PHP.B viral vectors containing a beta-galactosidase reporter gene under control of a CMV promoter were then introduced to each well at a MOI ranging from 100,000 to 10. This transduction step was performed in 100 uL of serum-free DMEM for 2 h, followed by addition of 100 uL DMEM+20% FBS+1% P/S and incubation for 24 h. Beta-galactosidase expression was then determined using the Galacto-Star B-Galactosidase Reporter Gene Assay System (Thermo-Fisher Scientific #T1014) as per the manufacturer's Direct Lysis Protocol for Microplate Cultures. The Lysis step of this protocol was modified in that 40 uL of lysis buffer was used per well, and lysis was performed for 30 minutes. Luminescence detection was then performed on a SpectramaxM3 Luminescence Plate Reader. Data are representative of six independent experiments.

### HEK293 Antibody Inhibition Assay

The HEK293 Antibody Inhibition Assay was performed as described above in the HEK293 Transduction Assay with the following modifications or additions. Only the C57BL/6J and Balb/cJ Ly6a-IRES2-EGFP plasmids were used in transfection. Additionally, an antibody incubation step was added before AAV9-PHP.B transduction on Day 4 of the assay. In this step, D7 endotoxin-low and azide-free anti-LY6A antibody (Abeomics #31-2027) or IgG isotype control (Abcam #18450) were incubated with cells for 1 hr at 4° C. Antibodies were introduced in 50 uL serum-free DMEM at 100 nM and following this incubation, AAV9-PHP.B reporter vector was introduced in 50 uL serum-free DMEM at a MOI of 10,000. Plates were then returned to the 37° C. incubator, and the remainder of the assay proceeded as previously described. Data are representative of eight independent experiments.

### Quantification and Statistical Analysis

Vector genome copies in mice were analyzed using one-way ANOVA (Kruskal Wallis test) followed by Dunn's multiple comparisons test with alpha value of 0.05 (GraphPad Prism). For WES linkage analysis, trait associated variants were identified using a linear Wald test for quantitative traits (https://github.com/statgen/EPACTS). Only those variants with a p-value less than or equal to 5E-8 were considered significant. HEK 293 transduction efficiencies (Beta-galactosidase activity) were compared using a 2-way ANOVA followed by a mean-comparison test using Tukey's multiple comparison test (GraphPad Prism).

### Results

### BBB Permeability to AAV-PHP.B is Inherited as a Codominant Trait in Mice

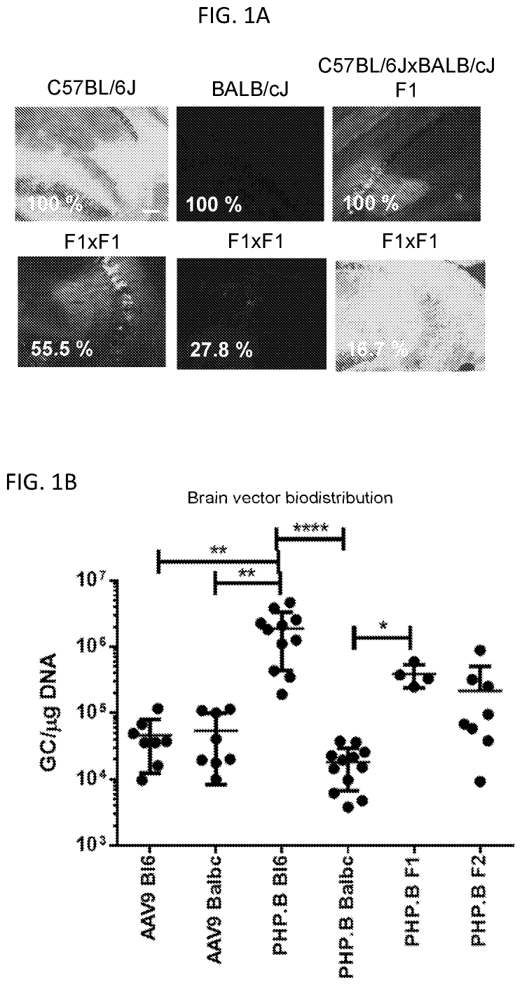

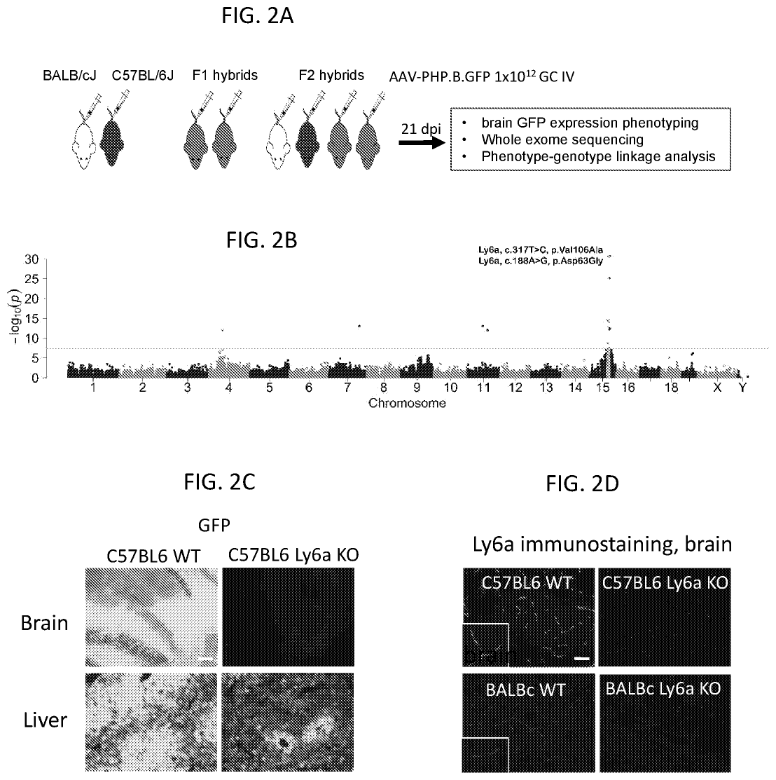

It was previously shown that IV administration of 1×10¹² genome copies (GC) of AAV-PHP.B carrying the GFP transgene resulted in widespread transduction of central nervous system (CNS) cells in C57BL/6J mice, but not BALB/cJ mice¹³. In contrast, direct administration of AAV-PHP.B into the CNS by intracerebroventricular injection, which bypasses the BBB, led to equally robust GFP expression in both C57BL/6J and BALB/cJ mouse brains (FIG. 5A and FIG. 5B). Therefore, it was concluded that cells of the CNS of both strains were susceptible to AAV-PHP.B brain transduction but that the increased efficiency of AAV-PHP.B in C57BL/6J mice was due to enhanced delivery across the BBB. It was hypothesized that the strain specific differences in AAV-PHP.B permeability of the BBB was caused by genetic variation in a single gene involved in BBB transport. To confirm this hypothesis CNS transduction was evaluated following IV administration of AAV-PHP.B in F1 and F2 progenies of C57BL/6J×BALB/cJ crossings. All F1 offsprings displayed intermediate CNS transduction compared to the parental strains; while the F2 generation showed a distribution of transduction with 55.5% intermediate, 16.7% high C57BL/6J-like, and 27.8% low BALB/cJ-like CNS transduction (FIG. 1A). This result was confirmed by qPCR-based quantification of vector genome copy numbers in the corresponding mouse brains (FIG. 1B). Based on phenotype distributions of F1 and F2, it was conclude that BBB permeability to PHP.B follows the Mendelian inheritance pattern of two codominant alleles at a single genomic locus. In contrast to AAV-PHP.B, we did not observe any strain-specific brain transduction for AAV9 (FIG. 1B).

### The Ly6a Gene is Linked to PHP.B Transduction Across the BBB

The pattern of inheritance of CNS transduction described in F1 and F2 progeny suggests that variation in a single gene may underlie the strain specific differences in this phenytype (i.e, high BBB permeability of AAV-PHP.B). Therefore, whole exome sequencing (WES) was conducted based on genotyping and genetic linkage analysis of 16 related mice to identify any causal mutations within coding regions of the mouse genome (FIG. 2A). Our analysis identified 135 unique mutations located within a ˜4.5 Mbp stretch of genomic DNA spanning the D3 and E3 karyotype bands of murine chromosome 15 that are most significantly associated (p=1.9E-31) with the observed phenotype (FIG. 2B). Functional variant annotation of the identified significant variants (p≤5E-8) revealed that missense mutations within the Ly6a, Ly6i, Rhophilin 1, and Riken cDNA 2010109103 genes are most significantly linked with high BBB permeability of AAV-PHP.B (Table 1). Based on the subcellular localization of the proteins encoded by these genes and their abundance in the brain according to public databases¹⁴, it was hypothesized that LY6A (also known as SCA-1), a GPI-anchored surface protein highly expressed in the brain microvasculature, was the causative protein. To test whether Ly6a is essential for highly efficient delivery of AAV-PHP.B across the BBB, AAV-PHP.B carrying the GFP transgene was IV injected in Ly6a knockout (Ly6a^{-/-}) mice in the C57BL/6J background and wild type controls. While PHP.B effectively transduced liver in Ly6a^{-/-} mice, minimal brain transduction was observed (FIG. 2C), indicating that LY6A is required for AAV-PHP.B transport across the BBB. Interestingly, polymorphism in the Ly6 gene cluster has been described previously, with two major haplotypes in inbred strains, Ly6^{a} (BALB/cJ-like) and Ly6^{b} (C57BL/6J-like). These two haplotypes differ with respect to several single nucleotide changes within the promotor region and two amino acid substitutions in the LY6A protein in (i.e., the Ly6^{a}-encoded proteins have Val106Ala and pAsp63Gly substitution relative to the Ly6^{b}). Analysis of brain tissue by immunohistochemistry with an antibody to LY6A revealed high level expression in microvascular endothelial cells of C57BL/6J animals that is substantially reduced in BALB/cJ animals (FIG. 2D) which confirms previous reports¹⁵. No expression of LY6A was detected in tissue from Ly6a^{-/-} mice confirming the specificity of the assay (FIG. 2D). Either the promoter mutations or those within the open reading frame of BALB/cJ LY6A could contribute to the drastically decreased LY6A expression on brain endothelium.

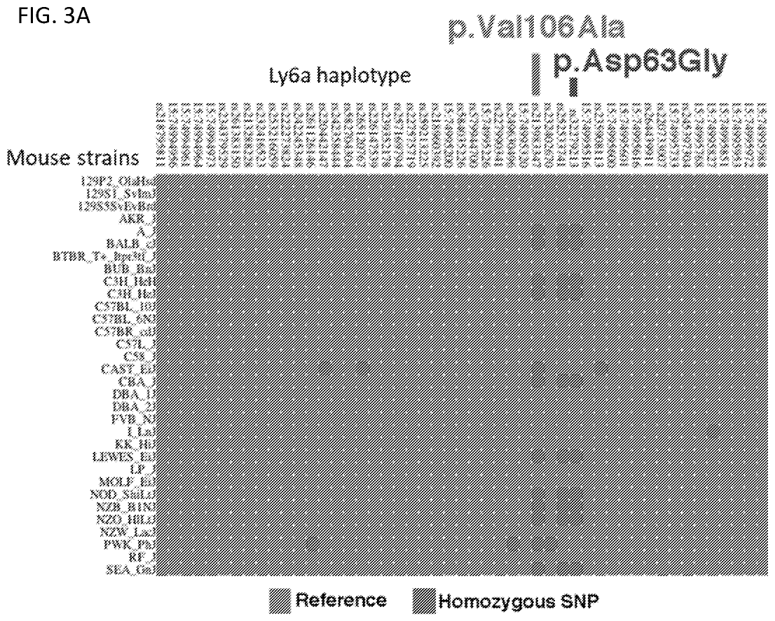

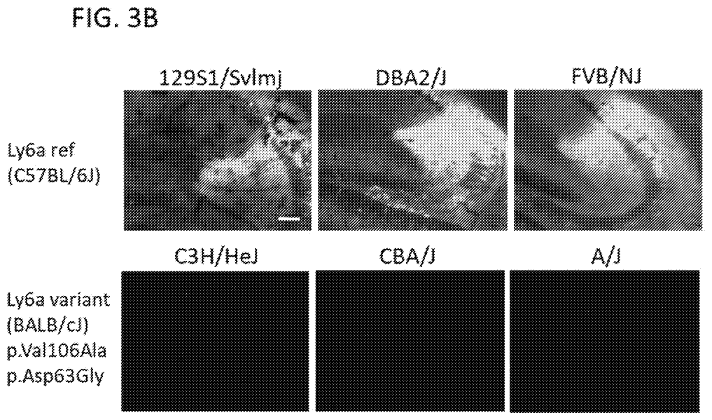

Based on these observations, it was hypothesized that strain-specific BBB permeability of AAV-PHP.B observed in C57BL/6J and BALB/cJ mice can be generalized to all stains of mice with Ly6^{b} and Ly6^{a} haplotypes. In support of this hypothesis, 6 additional inbred strains of mice were tested, 3 of which had the Ly6^{b} haplotype (129S1/Svlmj, DBA2/J, and FVB/NJ) and 3 the Ly6^{a} haplotype (C3H/HeJ, CBA/J and A/J) (FIG. 3A). Mice from each strain were infused IV with AAV-PHP.B expressing GFP and analyzed for CNS transduction. As predicted, high level CNS transduction directly correlated with the Ly6^{b} (C57BL/6J-like) haplotype (FIG. 3B). Taken together, it was concluded that the Ly6a gene variants are linked to PHP.B transduction across the BBB.

TABLE 1
Variants linked with BBB permeability to AAV-PHP.B vector in mice

| Variant | | | | | | |
|---|---|---|---|---|---|---|
| Type | AA Mutation | Chromosome | Position | Gene ID | Karyotype Band | P-Value |
| missense_variant | p.Val95Ile | 15 | 74879901 | 2010109I03Rik | D3 | 1.90E−31 |
| missense_variant | p.Lys68Arg | 15 | 74980116 | Ly6i | D3 | 1.90E−31 |
| missense_variant | p.Ala6Glu | 15 | 74983043 | Ly6i | D3 | 1.90E−31 |
| missense_variant | p.Val106Ala | 15 | 74995350 | Ly6a | D3 | 1.90E−31 |
| missense_variant | p.Asp63Gly | 15 | 74995479 | Ly6a | D3 | 1.90E−31 |
| &splice_region_variant | | | | | | |
| missense_variant | p.Ser527Gly | 15 | 75713294 | Rhpn1 | D3 | 1.90E−31 |
| missense_variant | p.Glu22Gly | 15 | 75048447 | Ly6c1 | D3 | 3.27E−08 |
| splice_donor_variant | N/A | 15 | 75372840 | Gm3142 | D3 | 3.27E−08 |
| &intron_variant | | | | | | |
| missense_variant | p.Glu79Asp | 15 | 71806227 | Maf1 | D3 | 7.91E−08 |
| missense_variant | p.Leu56Ser | 15 | 71820059 | Bop1 | D3 | 7.91E−08 |
| missense_variant | p.Thr133Ala | 15 | 71821959 | Adck5 | D3 | 7.91E−08 |
| missense_variant | p.Met1352Leu | 15 | 72852493 | Tonsl | D3 | 7.91E−08 |
| missense_variant | p.Thr1086Met | 15 | 74539785 | Tonsl | D3 | 7.91E−08 |
| missense_variant | p.Tyr909Cys | 15 | 76352871 | Tonsl | D3 | 8.94E−08 |
| frameshift_variant | p.Ser360fs | 15 | 76474848 | Cyhr1 | D3 | 8.94E−08 |
| missense_variant | p.Gln308Arg | 15 | 76593485 | Kifc2 | D3 | 8.94E−08 |
| missense_variant | p.Leu527Met | 15 | 76626339 | Recql4 | D3 | 8.94E−08 |
| missense_variant | p.Asp503Glu | 15 | 76630928 | Lrrc24 | D3 | 8.94E−08 |
| missense_variant | p.Leu78Phe | 15 | 76632792 | Zfp251 | D3 | 8.94E−08 |
| missense_variant | p.Glu72Asp | 15 | 76647499 | Zfp7 | D3 | 8.94E−08 |
| missense_variant | p.Leu280Phe | 15 | 76662816 | Apol11a | E1 | 8.94E−08 |
| missense_variant | p.Asn290Lys | 15 | 76707129 | Apol11a | E1 | 8.94E−08 |
| missense_variant | p.Lys293Glu | 15 | 76715429 | Apol11a | E1 | 8.94E−08 |
| missense_variant | p.Tyr297Asp | 15 | 76869852 | Apol11a | E1 | 8.94E−08 |
| missense_variant | p.Arg308Gln | 15 | 76888316 | Apol11a | E1 | 8.94E−08 |
| missense_variant | p.Thr167Asn | 15 | 77517152 | Apol7c | E1 | 8.94E−08 |
| missense_variant | p.Ala137Thr | 15 | 77517184 | Apol7c | E1 | 8.94E−08 |
| missense_variant | p.Glu130Gly | 15 | 77517191 | Apol7c | E1 | 8.94E−08 |
| missense_variant | p.Ile128Leu | 15 | 77517203 | Apol7c | E1 | 8.94E−08 |
| missense_variant | p.Gln73Lys | 15 | 77517237 | Apol7c | E1 | 8.94E−08 |
| missense_variant | p.Glut65Val | 15 | 77526245 | Apol10b | E1 | 8.94E−08 |
| missense_variant | p.Glut65Val | 15 | 77526336 | Apol10b | E1 | 8.94E−08 |
| missense_variant | p.Phe130Ser | 15 | 77526356 | Apol1b | E1 | 8.94E−08 |
| missense_variant | p.Asp109Glu | 15 | 77526363 | Apol10b | E1 | 8.94E−08 |
| missense_variant | p.Val50Ala | 15 | 77528832 | Apol11b | E1 | 8.94E−08 |
| missense_variant | p.Ile30Arg | 15 | 77585482 | Apol11b | E1 | 8.94E−08 |
| disruptive_ inframe_deletion | p.Thr220del | 15 | 77585482 | Phf21b | E2 | 8.94E−08 |
| missense_variant | p.Lys1461Glu | 15 | 77585587 | Col22a1 | D3 | 8.94E−08 |
| missense_variant | p.Gln1304Leu | 15 | 77585649 | Col22a1 | D3 | 8.94E−08 |
| missense_variant | p.Ser1286Pro | 15 | 77637947 | Col22a1 | D3 | 8.94E−08 |
| missense_variant | p.Gln850His | 15 | 77638007 | Trappc9 | D3 | 8.94E−08 |
| missense_variant | p.Arg379His | 15 | 84803488 | Adgrb1 | D3 | 8.94E−08 |

AAV-PHP.B and AAV-PHP.eB Bind to the LY6A Protein with High Affinity

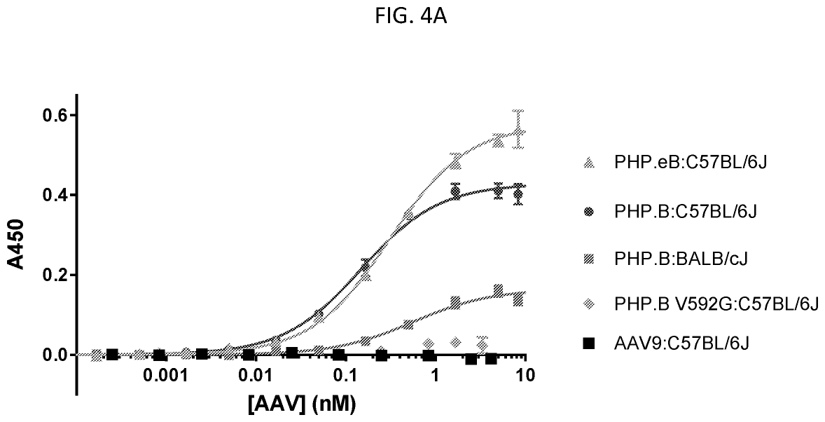

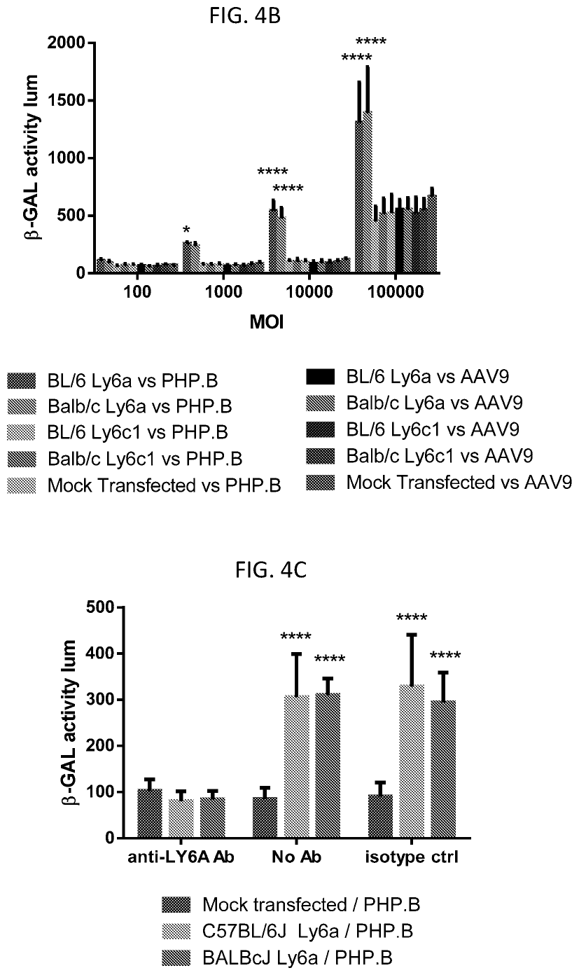

ELISA assays were developed to evaluate the potential for direct interactions between LY6A variants and AAV capsids. Expression cassettes containing GPI-anchor truncated versions of LY6A for both C57BL/6J and BALB/cJ variants were constructed to allow for isolation of soluble versions of the respective recombinant proteins. The ELISA assay was developed by analyzing binding of AAV particles to recombinant LY6A proteins bound to the ELISA well (FIG. 4A). More AAV.PHP.B bound to C57BL/6J LY6A than to BALB/cJ LY6A. No binding was detected between AAV9 and the LY6A variants (FIG. 4A). Despite less signal intensity with AAV-PHP.B to BABL/cJ LY6A, there was residual binding of relatively high affinity. In fact, both data sets fit sub-nanomolar binding isotherms well (0.07 nM and 0.28 nM for C57BL/6J and BALB/cJ variants respectively). We suspect this affinity represents the avid engagement of multiple, immobilized Ly6a proteins with AAV-PHP.B vectors. The reduction of BBB transport of AAV-PHP.B in BABL/cJ may be caused by lower binding to LY6A and reduced expression of LY6A.

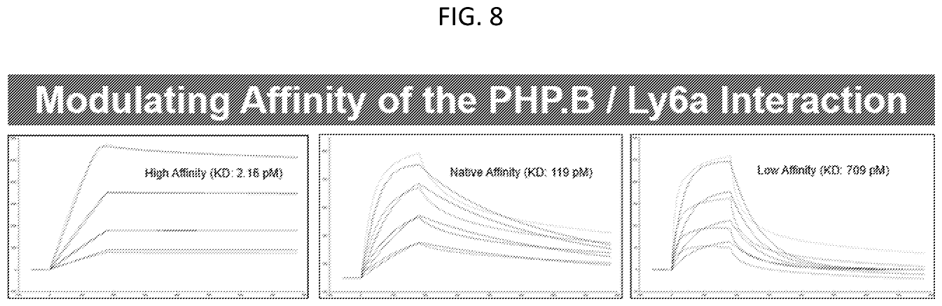

Valine 592 in the AAV9-PHP.B capsid, which falls in the middle of the 588-TLAVPFK peptide insertion, was subjected to saturating mutagenesis. Each vector variant was purified and individually tested for binding affinity to LY6A using SPR. Biacore sensograms identified high affinity, native affinity, and low affinity variants of AAV9-PHP.B (FIG. 8A-FIG. 8C).

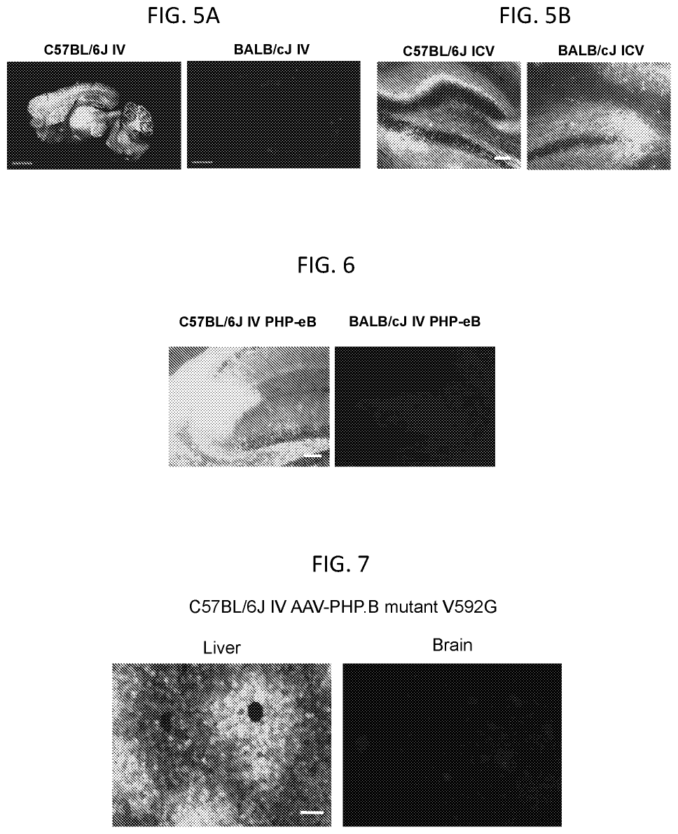

A variant capsid AAV-PHP.B V592G with a single amino-acid mutation in the 7 amino acid loop of AAV-PHP.B (valine to glycine on VP1 position 592 in the 4^{th} residue of the 7 amino acid insert) was generated. The expression profile after IV injection of a GFP expressing version of this mutant capsid resembled that of AAV9 but not AAV-PHP.B (e.g., high liver transduction but little CNS transduction) (FIG. 6). Interestingly, neither AAV9 nor AAV-PHP.B V592G bind detectibly to LY6A (FIG. 4A), showing direct link between capsid interaction with LY6A in vitro and the ability to cross the BBB in vivo.

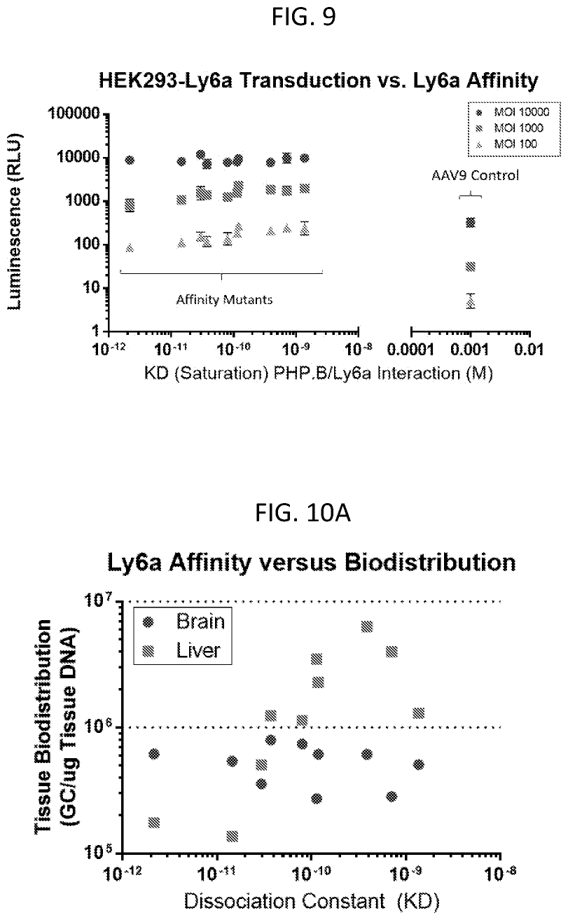

The ability of LY6A to enhance internalization of AAV capsids was then studied. In this assay, full-length Ly6a genes were transiently transfected into HEK293 cells that were subsequently incubated with low and high affinity capisd, including AAV-PHP.B or AAV9 expressing lacZ. In this assay transcduction of lacZ is a proxy for internalization. Transduction as measured by ß-galactosidase activity in cell lysates was achieved in a dose dependent manner with AAV-PHP.B incubated with cells expressing C57BL/6J or BALB/cJ LY6A proteins (FIG. 4B). No transduction over background was achieved with AAV9 on cells expressing any version of LY6A or with AAV-PHP.B on cells expressing the unrelated GPI-anchored protein LY6C1 (FIG. 4B). Low affinity variants show modest (2-fold) increase in transduction efficiency relative to high affinity variants at low MOI. Any level of binding to the Ly6a receptor is sufficient to boost transduction efficiency >10 fold relative to AAV9 (FIG. 9). Enhanced transduction of PHP.B on cells expressing C57BL/6J or BALB/cJ LY6A proteins was moreover prevented by preincubation of cells with an anti-LY6A rat monoclonal antibody (and not by preincubation with an isotype control), further reinforcing the hypothesis of a co-receptor or receptor role for LY6A (FIG. 4C).

A second generation version of AAV-PHP.B called AAV-PHP.eB was recently described with even greater transduction of CNS in C57BL/6J mice following IV injection. This variant was studied to determine if LY6A remains the primary determinant of BBB permeability. CNS transduction was observed in C57BL/6J mice but not BALB/cJ mice following IV injection of AAV-PHP.eB, as was observed with AAV-PHP.B (FIG. 7).

AAV-PHP.eB bound to C57BL/6J LY6A with a slightly higher signal than that achieved with AAV-PHP.B (FIG. 4A). It was concluded that interactions with LY6A remain critical to the neurotropic properties of both AAV-PHP.B and AAV-PHP.eB.

### Impact of LY6A Binding Affinity on Biodistribution

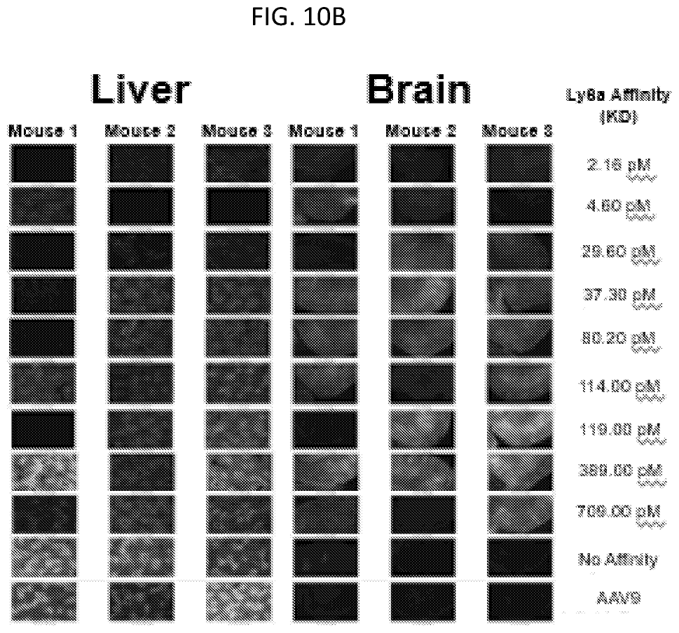

AAV9-PHP.B affinity variants were prepared with a CB7-eGFP reporter gene and tested in C57BL/6 mice at an IV dose of 1e12 gc/mouse. Mice were sacrificed 21 days post injection, and organs were harvested for histology and biodistribution. Ly6a affinity shows a negative correlation with liver biodistribution, wherein vectors with the strongest affinity for Ly6a show decreased localization to liver. This trend is not observed for brain biodistribution. All vectors with affinity for the LY6A receptor demonstrate comparable levels of localization to brain tissue (FIG. 10A). Liver expression of eGFP tracks with biodistribution, where variants with low affinity for Ly6a demonstrate increased localization to and expression in liver tissue. Brain histology indicates that although high and low affinity vectors have similar biodistribution in brain tissue, vectors with low to moderate affinity for LY6A have improved expression (FIG. 10B).

### Valency of PHP.B Peptide Presentation and Effects on Cellular Transduction

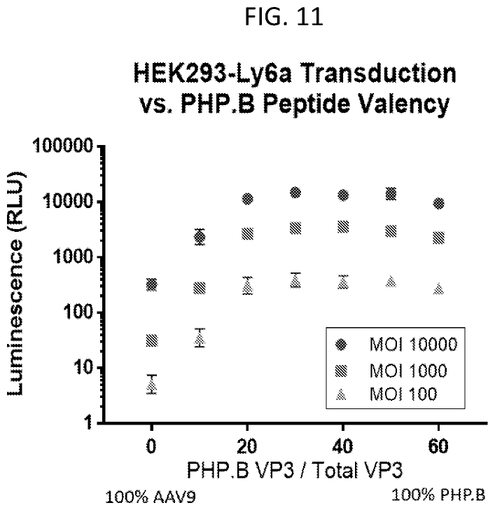

Chimeric capsids were produced by altering the ratios of plasmids encoding either the AAV9 or AAV9-PHP.B capsid gene used during vector production. Transduction efficiency of each chimeric variant was quantified by expression of beta-galactosidase in HEK293 cells stably expressing Ly6a. Capsids presenting the PHP.B peptide in a 1:3 PHP.B, 2:3 AAV9 ratio, when compared with capsids presenting 100% PHP.B peptide, transduced cells equivalently. Modulating valency of PHP.B peptide presentation also produces a graded response in transduction, whereas modulating peptide affinity (FIG. 11) produces more of a binary response in transduction.

### Multivalent Presentation of LY6A Domain is Required for Binding Between Soluble LY6A and PHP.B

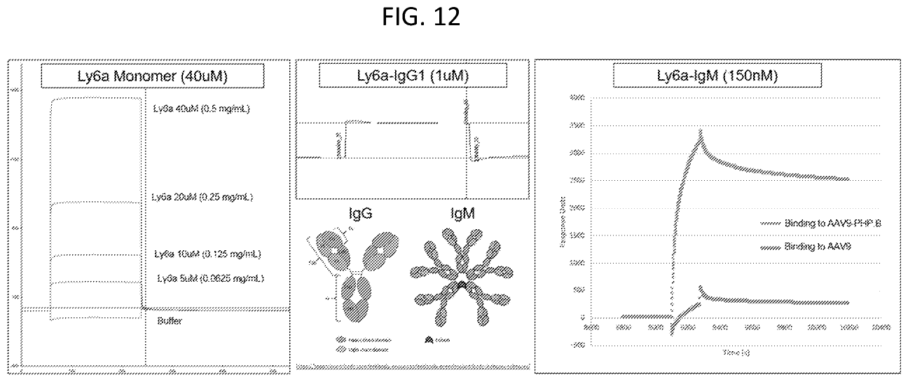

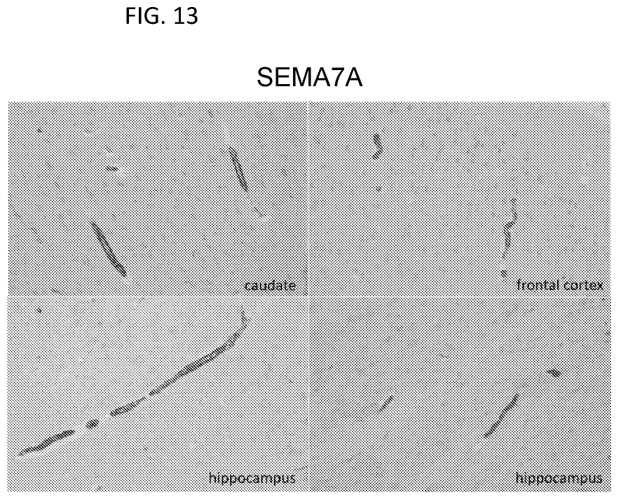

Previous binding and affinity SPR data were generated with LY6A immobilized on the surface of the sensor chip and AAV9-PHP.B in solution over the sensor surface. This orientation enables the vector to engage multiple surface receptors, strengthening the binding interaction through avidity effects. In the reverse orientation, monomeric LY6A is unable to bind to surface-immobilized AAV9-PHP.B, even at concentrations up to 40 uM. When LY6A is expressed as a dimer, fused to the N-terminus of an IgG1-Fc domain, it is still unable to bind surface-immobilized AAV9-PHP.B at concentrations of 1 uM. When LY6A is expressed as an N-terminal fusion to the IgM-Fc domain, up to 12 copies of the Ly6a domain can be presented on a single molecule. With this construct, specific binding to AAV9-PHP.B, but not AAV9 can be observed (FIG. 12).

### Discussion

As the technology of gene transfer has progressed, so has our understanding of key drivers of performance such as transduction efficiency and host vector responses. Some of these translational studies have enhanced our understanding of related fundamental biological processes. The ability of AAV vectors to transduce cells in the CNS of all mammals in a deliberate and dose dependent manner has revolutionized the study of neurobiology through the use of tools such as optogenetics (reviewed in¹⁶). In studies designed to blunt adaptive immune responses to in vivo gene transfer with adenoviruses, it was discovered the mechanism by which engagement of CD40 with CD40L activates T cells¹⁷. The story described in this paper is another example of translational work illuminating a new biological principle of broader impact than that related to gene therapy.

The isolation of the AAV9 variant AAV.PHP.B was heralded as potentially transforming the treatment of neurological diseases. The efficiency with which it could transduce CNS cells after IV injection in C57BL/6J mice was at least 20-fold higher than what had previously achieved and, if translated to primates, would enable the development of treatments across a broad array of neurological diseases¹⁸. Our initial evaluation of AAV-PHP.B led to the serendipitous discovery that its BBB permeability was limited to some strains of mice such as C57BL/6J¹³. The substantial difference in transduction biology between two related but genetically distinct strains of mice such as C57BL/6J and BALB/cJ allowed us to use a classic genetic linkage approach to determine that only one gene determines this dramatic strain difference in BBB permeability and that it encodes the GPI-anchored protein LY6A.

LY6A, also called Sea-1, was discovered more than forty years ago as an antigen upregulated on activated lymphocytes. It is commonly used to enrich adult murine hematopoietic stem cells (HSCs) and is also expressed on stem cells, progenitors, and differentiated cell types in a wide variety of tissues and organs (reviewed in⁹). It is fascinating that despite its common use in stem cell biology research, no ligand(s) of LY6A have been identified¹⁹⁻²¹. While its physiological functions are also unclear, the study of Ly6a-null mice suggest a role in T-cell proliferation downregulation²², hematopoiesis lineage regulation, HSCs engraftment and homing¹⁰, mesenchymal stem cells self-renewal, and bone formation^{23,24}. Here, for the first time a ligand for LY6A (i.e., AAV9 capsid variant AAV.PHP.B) was identified, and a direct link between binding of this ligand to LY6A, and its ability to cross the BBB was demonstrated. LY6A is highly expressed in brain microvasculaturel^{14,15,25}, and the Ly6^{b} haplotype was previously shown to be linked with wild-type Mouse Adenovirus 1-induced lethal encephalitis^{26,27}. In this context, our results suggest that this murine GPI-anchored protein could play a role in viral interaction and transcytosis at the BBB.

Studies of other GPI-anchored proteins suggest mechanisms by which LY6A could enhance BBB permeability of AAV vectors. GPI-anchored proteins often are localized to lipid rafts, which are dynamic microdomains within the plasma membrane that are enriched in cholesterol, sphingolipids, and a specific set of key-signaling molecules such as receptors and protein tyrosine kinases. Apical to basolateral delivery of raft-associated GPI-anchored proteins occurs via a transcytotic pathway in polarized cells²⁸. Furthermore, transcytosis of macromolecules at the BBB is known to occur in part via tightly regulated caveolae-associated lipid rafts²⁹. Some GPI-anchored proteins, and lipid rafts in general, play a role in the cell entry and exit of virus particles (reviewed in³⁰). Interestingly, Group B coxsackieviruses cross epithelial barriers through a 3 step mechanism involving 1/interaction with the apically localized GPI-anchored protein CD55/decay-accelerating factor (DAF), 2/CD55 clustering and activation of Ab1 kinase driving Rac-dependent actin reorganization, and 3/translocation of viral particles to lateral tight junctions where they engage their receptor CAR and undergo endocytosis³¹. In this model, GPI-anchored proteins allow initial capture of viral particles, and trigger transport of the virus to a receptor buried in the tight-junctions area. In the case of AAV-PHP.B transduction across the BBB, further experiments are needed to determine whether LY6A is a co-receptor facilitating colocalization of the viral capsids with other factors, or if it acts as the main receptor through cross-linking activated endocytosis.

Experimental data suggest that AAV9 is able to cross the BBB without compromising its integrity through transendothelial trafficking to the basolateral compartment³². The efficiency with which this occurs, however, is at least 20-fold lower than what is achieved with AAV-PHP.B in C57BL/mice⁸. Other AAV serotypes such as AAV5 can cross epithelial and endothelial barriers in vitro through transcytosis, a phenomenon blocked by tannic acid or filipin³³, two chemicals that interfere with the transport of GPI-anchored proteins. Interestingly, it was suggested that recombinant AAV2 uses the clathrin-independent carriers/GPI-anchored-protein-enriched endosomal compartment (CLIC/GEEC) endocytic pathway as a major transduction route³⁴. Those studies, conducted by independent groups, together with the work reported here support a central role of GPI-enriched lipid rafts in AAV transcytosis and/or transduction. Our results are the first to show that a GPI-anchored protein can act as a co-receptor or receptor for AAV vectors.

### Example 2: Engineered Capsids that Bind GPI-Anchored Proteins to Mediate Transduction Across the BBB

GPI-anchored proteins expressed on BBB endothelial cells can be hijacked for improving the delivery of biotherapeutics. Several groups, however, have failed to demonstrate increased CNS transduction following IV injection of AAV-PHP.B in non-human primates^{13,35}, which is probably explained by the absence of a LY6A homolog in primates³⁶. In fact, the only animal models that show enhanced BBB permeability of AAV.PHP.B are the ones with similar genetic backgrounds as the model in which it was selected (i.e., C57BL/6J mice), illustrating how the method of selecting novel capsid variants could limit the utility of candidate capsids. Therefore, it is also productive in the development of human gene therapy vectors and other protein therapeutics to evaluate populations of variants for binding to GPI-anchored proteins expressed on endothelial cells derived from primates.

While LY6A does not have a human ortholog, other LY6 family members and other GPI-anchored proteins are highly expressed on human brain endothelium (Table 2, source ISH/IHC human protein atlas www.proteinatlas.org) or have increased brain endothelium expression based on murine single-cell RNAseq data³ (Table 3). Moreover, some human LY6 proteins such as LY6E have been linked with neurotropic/endotheliotropic flavivirus infections. Accordingly, AAV vectors and capsids can be engineered to interact with GPI-anchored proteins expressed on human brain endothelium to mediate efficient transduction and delivery across the BBB.

TABLE 2
Human GPI-anchored proteins with brain endothelial expression
(protein atlas from human tissues www.proteinatlas.org)

| | | Expression level on |
|---|---|---|
| | | brain endothelium from |
| Gene | | protein atlas |
| name | Comment | (IHC labeling) |
| GFRA3 | GDNF receptor family - expressed on brain | 3 |
| | endothelium and neurons | |
| | Natural ligand Artemin + human Ab CDR in | |
| | patents | |
| | https://patents.google.com/patent/WO2014031712A1 | |
| ALPL | Tissue nonspecific alkaline phosphatase - | 2 to 3 |
| | unknown physiological function. Note: some | |
| | slides with very strong staining on endothelial | |
| | cells on the protein atlas | |
| BST2 | Aka tetherin (CD317). Also expressed in | 2 |
| | mature B cells; monocyte/macrophages, pDC, | |
| | IFNG inducible in other cell types. Known to | |
| | interact with several enveloped viruses. Natural | |
| | ligand ILT7 | |
| EFNA5 | Ephrin. Binds to EphA Eph receptors. Induces | 2 |
| | compartmentalized signaling within a | |
| | caveolae-like membrane microdomain when | |
| | bound to the extracellular domain of its | |
| | cognate receptor. | |
| NT5E | Aka CD73. Hydrolyzes extracellular AMP to | 2 |
| | adenosine. Expressed on lymphocytes, | |
| | Overexpressed in cancer cells | |
| DPEP2 | Membrane bound dipeptidase - hydrolyze | 2 |
| | leukotriene D4 and others | |
| GPC1 | Glypican-1; Cell surface heparan sulfate | 2 |
| | proteoglycan. variable number of heparan | |
| | sulfate chains. Interacts with SLIT2. Involved | |
| | in the misfolding of prion | |
| LYPD5 | (Ly6 family) Involved in laminin binding | 2 |
| GPC6 | Cell surface heparan sulfate proteoglycan | 2 |
| | Glypican-6. Putative coreceptor for growth | |
| | factors, ECM proteins . . . | |
| CD14 | Innate immune response: Coreceptor with | |
| | TLR4 for the detection of LPS | |
| CA4 | Carbonic anhydrase enzyme expressed on the | 2 |
| | luminal surface of pulmonary and other | |
| | capillaries. Interacts with Band3 anion | |
| | transporter | |
| GPC5 | Glypican 5. Cell surface heparan sulfate | 2 |
| | proteoglycan | |
| CD59 | (Ly6 family) Aka MAC-inhibitory protein. | 2 |
| | Complement activation regulation. Viruses | |
| | such as HIV, human cytomegalovirus and | |
| | vaccinia incorporate host cell CD59 into their | |
| | own viral envelope to prevent lysis by | |
| | complement. | |
| TFPI | Coagulation pathway: Tissue factor pathway | 2 |
| | inhibitor: interacts and inhibits factor Xa | |
| EFNA1 | Cell surface GPI-bound ligand for Eph | 2 |
| | receptors, a family of receptor tyrosine kinases | |
| | which are crucial for migration, repulsion and | |
| | adhesion during neuronal, vascular and | |
| | epithelial development. Binds promiscuously | |
| | Eph receptors residing on adjacent cells, | |
| | leading to contact-dependent bidirectional | |
| | signaling into neighboring cells. Plays an | |
| | important role in angiogenesis and tumor | |
| | neovascularization | |
| EFNA3 | Same family as EFNA1 and 5 | 2 |
| HYAL2 | Hyalurinidase - exists as a lysosomal enzyme | 2 |
| | and a GPI-anchored enzyme. Receptor for the | |
| | Jaagsiekte sheep retrovirus | |
| MELTF | Melanotransferrin (aba CD228) | 1 |
| | Has been proposed as a target to direct | |
| | adenovirus across the BBB: | |
| | https://www.ncbi.nlm.nih.gov/pubmed/17167498 | |
| ULBP2 | stress-induced ligand for NKG2D receptor | 1 |
| EFNA4 | Same family as EFNA1 and 5 | 1 |
| CNTN5 | Cell adhesion molecule, contactin family | 1 |
| BCAN | Brevican. lectican family of chondroitin sulfate | 1 |
| | proteoglycans that is specifically expressed in | |
| | the central nervous system. | |
| RECK | Expressed in many cancer cells, membrane- | 1 |
| | anchored glycoprotein may serve as a negative | |
| | regulator for matrix metalloproteinase-9, a key | |
| | enzyme involved in tumor invasion and | |
| | metastasis | |
| CFC1 | Member of EGF family | 1 |
| SEMA7A | Semaphoring 7a aka CD108, expressed on | 1 |
| | activated lymphocytes and on erythrocytes | |
| | (John Milton Hagen blood group). Receptor for | |
| | the Malaria Plasmodium | |

TABLE 3
Human GPI-anchored proteins with suspected brain
endothelial expression (murine RNAseq data)

| | | Mouse brain endothelial Cells |
|---|---|---|
| Gene.symbol | Comments | FPKM |
| PRNP | Prion protein (p27-30) (Creutzfeldt- | 297.7 |
| | Jakob disease, Gerstmann-Strausler- | |
| | Scheinker syndrome, fatal familial | |
| | insomnia) | |
| LY6E | Lymphocyte antigen 6 complex, | 286.5 |
| | locus E (Ly6 family). Linked with | NHP IHC and ISH (Peter Bell) |
| | entry of virus, including some | show weak endothelial |
| | endotheliotropic/neurotropic (West | expression - look mostly |
| | Nile, Dengue, Chickungunya) | neuronal. |
| | | No biopsy on the protein atlas |
| SEMA7A | Semaphorin 7A, GPI membrane | 192.1 |
| | anchor (John Milton Hagen blood | |
| | group) | |
| PRND | Prion protein 2 (dublet) | 142.2 |
| EFNA1 | Ephrin-A1 | 114.6 |
| PLAUR | Plasminogen activator, urokinase | 68.7 No |
| | receptor. Regulates cell surface | |
| | plasminogen activation - expressed | |
| | on neutrophils; involved in | |
| | neutrophil recruitment at | |
| | inflammatory sites. (Ly6 family) | |
| TFPI | Tissue factor pathway inhibitor | 57.2 |
| | (lipoprotein-associated coagulation | |
| | inhibitor) | |
| CD24A | CD24 or CD24A is a mucin-type | 29.9 |
| | glycosylphosphatidylinositol (GPI)- | |
| | anchored glycoprotein expressed on | |
| | the surface of B cells, granulocytes, | |
| | epithelial, neuronal, and muscle | |
| | cells, and on a range of tumor cells. | |
| BST2 | Bone marrow stromal cell antigen 2 | 26.2 |
| MMP25 | Matrix metallopeptidase 25 | 19.6 |
| HYAL2 | Hyaluronoglucosaminidase 2 | 16.4 |
| ART3 | ADP-ribosyltransferase 3 | 13.9 |
| LYPD1 | LY6/PLAUR domain containing 1 | 10.9 |
| | (Ly6 family). Lynx family of | |
| | neurotransmitter binding protein | |

### REFERENCES

1. Gao, G. et al. Clades of Adeno-associated viruses are widely disseminated in human tissues. J Virol 78, 6381-6388, doi:10.1128/JVI.78.12.6381-6388.2004 (2004).
2. Bell, C. L. et al. The AAV9 receptor and its modification to improve in vivo lung gene transfer in mice. J Clin Invest 121, 2427-2435, doi:10.1172/JCI57367 (2011).
3. Zincarelli, C., Soltys, S., Rengo, G. & Rabinowitz, J. E. Analysis of AAV serotypes 1-9 mediated gene expression and tropism in mice after systemic injection. Mol Ther 16, 1073-1080, doi:10.1038/mt.2008.76 (2008).
4. Duque, S. et al. Intravenous administration of self-complementary AAV9 enables transgene delivery to adult motor neurons. Mol Ther 17, 1187-1196, doi: 10.1038/mt.2009.71 (2009).
5. Bevan, A. K. et al. Systemic gene delivery in large species for targeting spinal cord, brain, and peripheral tissues for pediatric disorders. Mol Ther 19, 1971-1980, doi:10.1038/mt.2011.157 (2011).
6. Mendell, J. R. et al. Single-Dose Gene-Replacement Therapy for Spinal Muscular Atrophy. N Engl J Med 377, 1713-1722, doi:10.1056/NEJMoa1706198 (2017).
7. Gao, G. P. et al. Novel adeno-associated viruses from rhesus monkeys as vectors for human gene therapy. Proc Natl Acad Sci USA 99, 11854-11859, doi:10.1073/pnas.182412299 (2002).
8. Deverman, B. E. et al. Cre-dependent selection yields AAV variants for widespread gene transfer to the adult brain. Nat Biotechnol 34, 204-209, doi:10.1038/nbt.3440 (2016).
9. Holmes, C. & Stanford, W. L. Concise review: stem cell antigen-1: expression, function, and enigma. Stem Cells 25, 1339-1347, doi:10.1634/stemcells.2006-0644 (2007).
10. Bradfute, S. B., Graubert, T. A. & Goodell, M. A. Roles of Sca-1 in hematopoietic stem/progenitor cell function. Exp Hematol 33, 836-843, doi: 10.1016/j.exphem.2005.04.001 (2005).
11. Morcos, M. N. F. et al. SCA-1 Expression Level Identifies Quiescent Hematopoietic Stem and Progenitor Cells. Stem Cell Reports 8, 1472-1478, doi: 10.1016/j.stemcr.2017.04.012 (2017).
12. Park, J. W., Park, J. M., Park, D. M., Kim, D. Y. & Kim, H. K. Stem Cells Antigen-1 Enriches for a Cancer Stem Cell-Like Subpopulation in Mouse Gastric Cancer. Stem Cells 34, 1177-1187, doi:10.1002/stem.2329 (2016).
13. Hordeaux, J. et al. The Neurotropic Properties of AAV-PHP.B Are Limited to C57BL/6J Mice. Mol Ther 26, 664-668, doi:10.1016/j.ymthe.2018.01.018 (2018).
14. Zhang, Y. et al. An RNA-sequencing transcriptome and splicing database of glia, neurons, and vascular cells of the cerebral cortex. J Neurosci 34, 11929-11947, doi: 10.1523/JNEUROSCI. 1860-14.2014 (2014).
15. Cray, C., Keane, R. W., Malek, T. R. & Levy, R. B. Regulation and selective expression of Ly-6A/E, a lymphocyte activation molecule, in the central nervous system. Molecular Brain Research 8, 9-15 (1990).
16. Betley, J. N. & Sternson, S. M. Adeno-associated viral vectors for mapping, monitoring, and manipulating neural circuits. Hum Gene Ther 22, 669-677, doi: 10.1089/hum.2010.204 (2011).
17. Yang, Y. & Wilson, J. M. CD40 ligand-dependent T cell activation: requirement of B7-CD28 signaling through CD40. Science 273, 1862-1864 (1996).
18. Deverman, B. E., Ravina, B. M., Bankiewicz, K. S., Paul, S. M. & Sah, D. W. Y. Gene therapy for neurological disorders: progress and prospects. Nat Rev Drug Discov, doi: 10.1038/nrd.2018.158 (2018).
19. Penvose, A. & Westerman, K. A. Sca-1 is involved in the adhesion of myosphere cells to alphaVbeta3 integrin. Biol Open 1, 839-847, doi:10.1242/bio.20121222 (2012).
20. English, A., Kosoy, R., Pawlinski, R. & Bamezai, A. A Monoclonal Antibody Against the 66-kDa Protein Expressed in Mouse Spleen and Thymus Inhibits Ly-6A.2-Dependent Cell-Cell Adhesion. The Journal of Immunology 165, 3763-3771, doi: 10.4049/jimmunol.165.7.3763 (2000).
21. Pflugh, D. L., Maher, S. E. & Bothwell, A. L. M. Ly-6 Superfamily Members Ly-6A/E, Ly-6C, and Ly-6I Recognize Two Potential Ligands Expressed by B Lymphocytes. The Journal of Immunology 169, 5130-5136, doi: 10.4049/jimmunol.169.9.5130 (2002).
22. Stanford, W. L. et al. Altered Proliferative Response by T Lymphocytes of Ly-6A (Sca-1) Null Mice. J. Exp. Med. 186, 705-717 (1997).
23. Holmes, C. et al. Longitudinal analysis of mesenchymal progenitors and bone quality in the stem cell antigen-1-null osteoporotic mouse. J Bone Miner Res 22, 1373-1386, doi: 10.1359/jbmr.070604 (2007).
24. Bonyaldi, M. et al. Mesenchymal progenitor self-renewal deficiency leads to age-dependent osteoporosis in Sca-1Ly-6A null mice. Proc Natl Acad Sci USA 100, 5840-5845 (2003).
25. Ma, X., Robin, C., Ottersbach, K. & Dzierzak, E. The Ly-6A (Sca-1) GFP Transgene is Expressed in all Adult Mouse Hematopoietic Stem Cells. Stem Cells 20, 514-521 (2002).
26. Spindler, K. R. et al. The major locus for mouse adenovirus susceptibility maps to genes of the hematopoietic cell surface-expressed LY6 family. J Immunol 184, 3055-3062, doi:10.4049/jimmunol.0903363 (2010).
27. Stier, M. T. & Spindler, K. R. Polymorphisms in Ly6 genes in Msq1 encoding susceptibility to mouse adenovirus type 1. Mamm Genome 23, 250-258, doi:10.1007/s00335-011-9368-9 (2012).
28. Polishchuk, R., Di Pentima, A. & Lippincott-Schwartz, J. Delivery of raft-associated, GPI-anchored proteins to the apical surface of polarized MDCK cells by a transcytotic pathway. Nat Cell Biol 6, 297-307, doi:10.1038/ncb1109 (2004).
29. Andreone, B. J. et al. Blood-Brain Barrier Permeability Is Regulated by Lipid Transport-Dependent Suppression of Caveolae-Mediated Transcytosis. Neuron 94, 581-594 e585, doi:10.1016/j.neuron.2017.03.043 (2017).
30. Metzner, C., Salmons, B., Gunzburg, W. H. & Dangerfield, J. A. Rafts, anchors and viruses—a role for glycosylphosphatidylinositol anchored proteins in the modification of enveloped viruses and viral vectors. Virology 382, 125-131, doi:10.1016/j.virol.2008.09.014 (2008).
31. Coyne, C. B. & Bergelson, J. M. Virus-induced Abl and Fyn kinase signals permit coxsackievirus entry through epithelial tight junctions. Cell 124, 119-131, doi:10.1016/j.cell.2005.10.035 (2006).
32. Merkel, S. F. et al. Trafficking of adeno-associated virus vectors across a model of the blood-brain barrier; a comparative study of transcytosis and transduction using primary human brain endothelial cells. J Neurochem 140, 216-230, doi:10.1111/jnc.13861 (2017).
33. Di Pasquale, G. & Chiorini, J. A. AAV transcytosis through barrier epithelia and endothelium. Mol Ther 13, 506-516, doi:10.1016/j.ymthe.2005.11.007 (2006).
34. Nonnenmacher, M. & Weber, T. Adeno-associated virus 2 infection requires endocytosis through the CLIC/GEEC pathway. Cell Host Microbe 10, 563-576, doi:10.1016/j.chom.2011.10.014 (2011).
35. Matsuzaki, Y. et al. Intravenous administration of the adeno-associated virus-PHP.B capsid fails to upregulate transduction efficiency in the marmoset brain. Neurosci Lett 665, 182-188, doi:10.1016/j.neulet.2017.11.049 (2018).
36. Loughner, C. L. et al. Organization, evolution and functions of the human and mouse Ly6/uPAR family genes. Hum Genomics 10, 10, doi:10.1186/s40246-016-0074-2 (2016).
37. Lock, M. et al. Rapid, simple, and versatile manufacturing of recombinant adeno-associated viral vectors at scale. Hum Gene Ther 21, 1259-1271, doi:10.1089/hum.2010.055 (2010).
38. Lock, M., Alvira, M. R., Chen, S. J. & Wilson, J. M. Absolute determination of single-stranded and self-complementary adeno-associated viral vector genome titers by droplet digital PCR. Hum Gene Ther Methods 25, 115-125, doi:10.1089/hgtb.2013.131 (2014).
39. Hwang, S., Kim, E., Lee, I. & Marcotte, E. M. Systematic comparison of variant calling pipelines using gold standard personal exome variants. Scientific reports 5, 17875, doi:10.1038/srep17875 (2015).
40. Poplin, R. et al. Scaling accurate genetic variant discovery to tens of thousands of samples. bioRxiv (2017).
41. Van der Auwera, G. A. et al. From FastQ data to high confidence variant calls: the Genome Analysis Toolkit best practices pipeline. Current protocols in bioinformatics 43, 11.10.11-33, doi:10.1002/0471250953.bi1110s43 (2013).
42. DePristo, M. A. et al. A framework for variation discovery and genotyping using next-generation DNA sequencing data. Nature genetics 43, 491-498, doi:10.1038/ng.806 (2011).
43. McKenna, A. et al. The Genome Analysis Toolkit: a MapReduce framework for analyzing next-generation DNA sequencing data. Genome research 20, 1297-1303, doi:10.1101/gr.107524.110 (2010).
44. Cingolani, P. et al. A program for annotating and predicting the effects of single nucleotide polymorphisms, SnpEff: SNPs in the genome of Drosophila melanogaster strain w1118; iso-2; iso-3. Fly 6, 80-92, doi:10.4161/fly.19695 (2012).

### Sequence Listing Free Text

The following information is provided for sequences containing free text under numeric identifier <223>.

| SEQ ID NO: | |
|---|---|
| (containing free text) | Free text under <223> |
| 1 | <223> AAVPHP.B variant capsid |
| 2 | <223> AAVPHP.eB variant capsid |

All publications cited in this specification and US Provisional Application Nos. 62/769,652 and 62/914,035, filed Nov. 20, 2018 and Oct. 11, 2019, respectively, are incorporated herein by reference. Applicant hereby incorporates by reference the Sequence Listing filed herewith. This file is labeled “18-8634PCT_ST25.txt”. While the invention has been described with reference to particular embodiments, it will be appreciated that modifications can be made without departing from the spirit of the invention. Such modifications are intended to fall within the scope of the appended claims.

1. A composition comprising a recombinant AAV having a capsid which comprises a binding partner for a GPI-anchored blood-brain barrier (BBB) ligand and which is conjugated to an effector entity.

2. The composition of claim 1, wherein the ligand is Ly6E.

3. The composition of claim 1, wherein the ligand is selected from GRA3, ALPL, BST2, EFNA5, NT5E, DPEP2, GPC1, LYPD5, GPC6, CD14, CA4, GPC5, CD59, TFPI, EFNA1, EFNA3, HYAL2, MELTF, ULBP2, EFNA4, CNTN5, BCAN, RECK, CFC1, SEMA7A, PRNP, LY6E, PRND, PLAUR, CD24A, MMP25, ART3, LYPD1, PIBF1, CAPRIN1, GFRA3, GPIHBP1, MACF1, and SEC24B.

4. The composition claim 1, wherein the capsid is an empty capsid.

5. The composition claim 1, wherein the capsid further comprises an AAV vector genome encoding a heterologous gene.

6. The composition claim 1, wherein the effector entity is a peptide, nucleic acid, siRNA, antibody, antibody fragment, small molecule, lipid nanoparticle, or cytotoxic agent.

7. The composition of claim 1, wherein the AAV capsid and effector entity are conjugated via a linker.

8. A method for treatment of a neurological disease or disorder in a subject in need thereof comprising contacting the BBB of the subject with an AAV having a capsid which comprises a binding partner for a GPI-anchored BBB ligand and which is conjugated to an effector entity, wherein capsid binding to the GPI-anchored BBB ligand mediates transport of the effector entity across the BBB.

9. The method of claim 8, wherein the ligand is selected from Ly6E, GRA3, ALPL, BST2, EFNA5, NT5E, DPEP2, GPC1, LYPD5, GPC6, CD14, CA4, GPC5, CD59, TFPI, EFNA1, EFNA3, HYAL2, MELTF, ULBP2, EFNA4, CNTN5, BCAN, RECK, CFC1, SEMA7A, PRNP, LY6E, PRND, PLAUR, CD24A, MMP25, ART3, LYPD1, PIBF1, CAPRIN1, GFRA3, GPIHBP1, MACF1, and SEC24B.

10. The method of claim 8, wherein the neurological disease or disorder is selected from the group consisting of Alzheimer's disease (AD), stroke, dementia, muscular dystrophy (MD), multiple sclerosis (MS), amyotrophic lateral sclerosis (ALS), cystic fibrosis, Angelman's syndrome, Liddle syndrome, Parkinson's disease, Pick's disease, Paget's disease, cancer, a lysosomal storage disorder, and traumatic brain injury.

11. The method of claim 8, wherein the effector entity is a peptide, nucleic acid, siRNA, antibody, antibody fragment, small molecule, or cytotoxic agent.

12. The method of claim 8, wherein the AAV capsid is conjugated to the effector entity via a linker.

13. A co-therapy for reducing or inhibiting central nervous system (CNS) uptake of a gene therapy vector having an AAV capsid with a binding partner for a GPI-anchored BBB ligand comprising co-administering with the gene therapy vector an antibody or antibody fragment that binds the BBB ligand.

14. The method of claim 13, wherein the ligand is selected from Ly6E, GRA3, ALPL, BST2, EFNA5, NT5E, DPEP2, GPC1, LYPD5, GPC6, CD14, CA4, GPC5, CD59, TFPI, EFNA1, EFNA3, HYAL2, MELTF, ULBP2, EFNA4, CNTN5, BCAN, RECK, CFC1, SEMA7A, PRNP, LY6E, PRND, PLAUR, CD24A, MMP25, ART3, LYPD1, PIBF1, CAPRIN1, GFRA3, GPIHBP1, MACF1, and SEC24B.

15. A method of engineering an AAV capsid to target the CNS comprising
a) identifying an amino acid sequence encoding a peptide fragment that specifically binds a GPI-anchored BBB ligand, and
b) modifying an AAV HVRVIII site to express said amino acid sequence, wherein the engineered capsid binds a GPI-anchored BBB ligand.

16. The method of claim 15, wherein the ligand is selected from Ly6E, GRA3, ALPL, BST2, EFNA5, NT5E, DPEP2, GPC1, LYPD5, GPC6, CD14, CA4, GPC5, CD59, TFPI, EFNA1, EFNA3, HYAL2, MELTF, ULBP2, EFNA4, CNTN5, BCAN, RECK, CFC1, SEMA7A, PRNP, LY6E, PRND, PLAUR, CD24A, MMP25, ART3, LYPD1, PIBF1, CAPRIN1, GFRA3, GPIHBP1, MACF1, and SEC24B.

17. The method of claim 15, wherein the modified AAV is AAV1, AAV3B, or AAV9.

18. An engineered AAV capsid which binds a GPI-anchored BBB ligand obtained by the method of claim 15.

19-21. (canceled)
